(19) United States
(12) Patent Application Publication
(10) Pub. No.: US 2017/0182088 A1
(43) Pub. Date: Jun. 29, 2017

(54) METHOD AND APPARATUS FOR ADMINISTERING NITRIC OXIDE WITH SUPPLEMENTAL DRUGS

(71) Applicant: GENO LLC, Cocoa, FL (US)

(72) Inventors: Kurt A. Dasse, Needham, MA (US); Priscilla C. Petit, Orlando, FL (US); David H. Fine, Cocoa Beach, FL (US); Gregory Vasquez, Cocoa, FL (US)

(73) Assignee: GENO LLC, COCOA, FL (US)

(21) Appl. No.: 15/382,601

(22) Filed: Dec. 16, 2016

Related U.S. Application Data
(60) Provisional application No. 62/272,064, filed on Dec. 28, 2015.

(51) Int. Cl. A61K 33/00; A61K 45/06; A61K 31/506; A61K 9/00; A61K 31/4422

(52) U.S. Cl. CPC A61K 33/00; A61K 9/007; A61K 31/4422; A61K 31/506; A61K 45/06

(57) ABSTRACT

A method of providing a therapeutic composition includes administering a ROS reducing drug, calcium channel blocker, anti-fibrotic, anti-inflammatory, or anti-hypertensive drug and administering inhaled nitric oxide and reducing symptoms of oxidative stress and/or fibrosis in a patient.

This application claims priority under 35 U.S.C. §119(e) to U.S. Patent Application Ser. No. 62/272,064 filed on Dec. 28, 2015, which is hereby incorporated by reference in its entirety.

### TECHNICAL FIELD

The invention relates to administering nitric oxide with supplemental drugs.

### BACKGROUND

An antioxidant is a molecule that inhibits the oxidation of other molecules. Oxidation is a chemical reaction involving the loss of electrons or an increase in oxidation state. Oxidation reactions can produce free radicals. In turn, these radicals can start chain reactions. When the chain reaction occurs in a cell, it can cause damage or death to the cell. Antioxidants terminate these chain reactions by removing free radical intermediates, and inhibit other oxidation reactions. They do this by being oxidized themselves, so antioxidants are often reducing agents such as thiols, ascorbic acid (vitamin C), or polyphenols.

Although oxidation reactions are crucial for life, they can also be damaging; plants and animals maintain complex systems of multiple types of antioxidants, such as glutathione, vitamin C, vitamin A, and vitamin E as well as enzymes such as catalase, superoxide dismutase and various peroxidases. Insufficient levels of antioxidants, or inhibition of the antioxidant enzymes, cause oxidative stress and may damage or kill cells. Oxidative stress is damage to cell structure and cell function by overly reactive oxygen-containing molecules and chronic excessive inflammation. Oxidative stress seems to play a significant role in many human diseases, including cancers. The use of antioxidants in pharmacology is intensively studied, particularly as treatments for stroke and neurodegenerative diseases. For these reasons, oxidative stress can be considered to be both the cause and the consequence of some diseases.

Nitric oxide, also known as nitrosyl radical, is a free radical that is an important signalling molecule. For example, NO can cause smooth muscles in blood vessels to relax, thereby resulting in vasodilation and increased blood flow through the blood vessel. These effects can be limited to small biological regions since NO can be highly reactive with a lifetime of a few seconds and can be quickly metabolized in the body. NO can also bond to haemoglobin and be transmitted peripherally to the brain, other end organs, and to the microvasculature to either act as a signalling molecule to induce neuroprotective effects, or to cause peripheral vasodilatation.

Some disorders or physiological conditions can be mediated by inhalation of nitric oxide. The use of low concentrations of inhaled nitric oxide can prevent, reverse, or limit the progression of disorders which can include, but are not limited to, acute pulmonary vasoconstriction, traumatic injury, aspiration or inhalation injury, fat embolism in the lung, acidosis, inflammation of the lung, adult respiratory distress syndrome, acute pulmonary edema, acute mountain sickness, post cardiac surgery acute pulmonary hypertension, persistent pulmonary hypertension of a newborn, perinatal aspiration syndrome, haline membrane disease, acute pulmonary thromboembolism, heparin-protamine reactions, sepsis, asthma and status asthmaticus, sickle cell anemia, acute renal injury or hypoxia. Nitric oxide can also be used to treat chronic pulmonary hypertension, bronchopulmonary dysplasia, chronic pulmonary thromboembolism and idiopathic or primary pulmonary hypertension or chronic hypoxia.

Generally, nitric oxide can be inhaled or otherwise delivered to the individual's lungs. Providing a therapeutic dose of NO could treat a patient suffering from a disorder or physiological condition that can be mediated by inhalation of NO or supplement or minimize the need for traditional treatments in such disorders or physiological conditions. Typically, the NO gas can be supplied in a bottled gaseous form diluted in nitrogen gas (N₂). Great care should be taken to prevent the presence of even trace amounts of oxygen (O₂) in the tank of NO gas because the NO, in the presence of O₂, can be oxidized to nitrogen dioxide (NO₂). Unlike NO, the part per million levels of NO₂ gas can be highly toxic if inhaled and can form nitric and nitrous acid in the lungs.

### SUMMARY

A method of providing a therapeutic composition includes administering a reactive oxygen species (ROS) reducing drug, administering inhaled nitric oxide, and reducing symptoms of oxidative stress and/or fibrosis in a patient.

This method can further include mixing a first gas including oxygen and a second gas including a nitric oxide-releasing agent within a receptacle to form a gas mixture, wherein the receptacle includes an inlet, an outlet and a reducing agent, and contacting the nitric oxide-releasing agent in the gas mixture with the reducing agent to generate nitric oxide.

In certain embodiments, the ROS reducing drug such as exogenous NO to minimize formation of endogenous NO, or to inhibit TGF-β, and connective tissue growth factor, or a ROS reducing agent.

In certain embodiments, the symptoms of oxidative stress include memory loss and/or brain fog.

In other embodiments, the symptoms of oxidative stress include fatigue.

In yet other examples, the symptoms of oxidative stress include muscle and/or joint pain.

In other examples, the symptoms of oxidative stress include decreased eye sight.

In yet other embodiments, the symptoms of oxidative stress include headaches and sensitivity to noise.

In certain embodiments, the symptoms of oxidative stress include susceptibility to infections.

In certain embodiments, the symptoms of oxidative stress include susceptibility of heart failure.

In certain embodiments, the symptoms of oxidative stress include renal injury.

In other embodiments, the nitric oxide-releasing agent is nitrogen dioxide.

In other examples, the method further includes delivering a hydrogen gas.

In certain examples, the hydrogen acts to eliminate peroxynitrite, thereby reducing adverse effects of nitric oxide.

In some examples, the ROS is a result of a disease. In other examples, the ROS is drug-induced.

In certain embodiments, the second gas includes an inert gas or oxygen.

In other embodiments, the concentration of nitric oxide in the gas mixture delivered is at least 0.01 ppm and at most 2 ppm.

In other embodiments, the patient is treated for symptoms of interstitial lung disease, oxygen-induced inflammation, cardiac ischemia, myocardial dysfunction, heart failure, ARDS, pneumonia, pulmonary embolism, COPD, emphysema, fibrosis, or mountain sickness due to high altitude.

In other embodiments, a method for providing a therapeutic composition includes identifying a mammal having or at risk of developing a haemolytic condition, positioning a mammal for nitric oxide treatment, administering exogenous nitric oxide, and reducing symptoms of hemolysis in the mammal.

The hemolysis can result from venom, e.g., from a snake, scorpion, sea anemones, or other venomous animal. The hemolysis can result from a bacterial infection. The hemolysis can also be caused by proteolysis. In the case of hemolytic activity, the venom creates localized anemia and, though unlikely, secondary renal failure. Proteolysis results in coagulation of the blood in the blood vessels, which, in turn, damages red blood cells and becomes a secondary cause of hemolysis.

In other embodiments, delivering the gas mixture including nitric oxide from the receptacle to the mammal includes passing the gas mixture through a delivery conduit located between the receptacle and a patient interface.

In yet other embodiments, the volume of the receptacle is greater than the volume of the delivery conduit.

In certain examples, the volume of the receptacle is at least two times the volume of the delivery conduit.

In yet other examples, delivering the gas mixture including nitric oxide from the receptacle to the mammal includes intermittently providing the gas mixture to the mammal.

In some embodiments, delivering the gas mixture including nitric oxide from the receptacle to the mammal includes pulsing the gas mixture.

In yet other embodiments, pulsing includes providing the gas mixture for one or more pulses of 1 to 6 seconds.

In other examples, the volume of the receptacle is greater than the volume of the gas mixture in a pulse.

In certain examples, the volume of the receptacle is at least twice the volume of the gas mixture in a pulse.

In yet other examples, the gas mixture is stored in the receptacle between pulses.

In other examples, the method further includes storing the gas mixture in the receptacle for a predetermined period of time, and wherein the predetermined period is at least 1 second.

In yet other examples, pulsing includes providing the gas mixture for two or more pulses and the concentration of nitric oxide in each pulse varies by less than 10%.

In certain other examples, pulsing includes providing the gas mixture for two or more pulses and the concentration of nitric oxide in each pulse varies by less than 10 ppm.

In other examples, the method includes communicating the first gas through a gas conduit to the receptacle and supplying the second gas into the gas conduit immediately prior to the receptacle.

In certain other examples, the method includes supplying the second gas at the receptacle.

In yet other examples, the method includes administering exogenous NO in an amount effective to modulate the hormesis characteristics of NO.

In certain examples, the nitric oxide is administered to neonates.

In other embodiments, the nitric oxide is administered to pediatric patients.

In yet other embodiments, the nitric oxide is administered to adults.

In certain examples, the use of exogenous NO may be used alone, in combination with, or as an alternative to, nitric oxide donors to modulate endogenous NO signalling within the initial cell to prevent oxidative stress, and to inhibit the inflammatory and profibrotic effects of TGF-B and CTGF.

In other examples, exogenous NO may be used to inhibit endogenous NO signalling to adjacent cells to prevent oxidative stress or activation of these other metabolic pathways. Low dose exogenous NO activates soluble guanylyl cyclase leading to vasodilation and a reduction in pulmonary hypertension. Riociguat also activates soluble guanylyl cyclase with resultant vasodilation. The combination of Riociguat plus NO has been shown to potentiate the vasodilatory effect of the administered NO. Therefore, the combination of administered NO with Riociguat may be used to gain a greater pulmonary hemodynamic effect reducing pressures and resistance compared to when each drug is used alone.

Prostacyclin-mediated effects lead to activation of GTP, cGMP mediated Protein Kinase(s), cGMP-gated cation channel(s), and possibly PDE (phosphodiesterase) mode of actions. Exogenous NO has demonstrated to have an added hemodynamic effect when administered in addition to prostacyclin therapy to further reduce pulmonary pressures. Therefore each drug may be used alone or in combination to reduce pulmonary pressure and pulmonary vascular resistance.

Exogenous NO may be used to control prostacyclin (and other systemic vasodilators/vasoconstrictors) to modulate gene expression, RNA transcription and translation to inhibit nitric oxide synthase synthesis (NOS 1, 2 and 3) to inhibit oxidative stress, inflammation and fibrosis

Exogenous NO may be administered to cause inhibition of the NOS family of synthesases to prevent oxidative stress

Exogenous NO may be administered to inhibit Ubiquitin targeting to prevent or minimize cellular apoptosis. Exogenous NO can also be provided as part of a therapeutic composition including a caspase regulator to modulate cellular apoptosis in a patient.

Exogenous NO may be administered to activate intracellular depolarization and hyperpolarization processes to turn on of gated channels that lead to vasodilation.

Exogenous NO may be administered to inhibit oxidative stress and other destructive metabolic pathways mediated by the cGMP molecule to protect the lungs, heart, kidneys, brain and remaining organs in the body.

Exogenous NO may be administered to control biochemical/metabolic pathways described above when NO is applied in combination with oral, inhaled and parenteral prostacyclin, and systemic vasodilators/vasoconstrictors) to prevent oxidative stress, inflammation and profibrotic pathophysiology.

In certain examples, a method of providing a therapeutic composition can be include identifying a mammal having or at risk of developing an ischemic condition, administering exogenous nitric oxide; and administering a drug with the nitric oxide to modulate remote ischemic conditioning pathway.

In certain embodiments, the exogenous NO can be administered over a 30 minute period at low dose effective to cause accumulation of hypoxia inducible factor(s) and PHDs to promote ROS signalling.

In other embodiments, the method can improve organ preservation by down regulating mitochondrial metabolic activity.

In certain embodiments, modulating hypoxia inducible factor(s) causes erythropoietin production to stimulate red cell production.

In other embodiments, the method further includes modulating a platelet derived growth factor pathway to reduce symptoms of fibrosis in a patient.

In certain embodiments, the NO can be provided through a cartridge that converts nitric oxide-releasing agents to NO. The cartridge can include an inlet, an outlet, and a reducing agent. The cartridge can be configured to utilize the whole surface area in converting nitric oxide-releasing agents to NO. The cartridge can have a length, width, and thickness, an outer surface, and an inner surface, and can be substantially cylindrical in shape. The cartridge can have aspect ratio of approximately 2:1, 3:1 or 4:1. The length can be, for example, one inch, two inches, three inches, four inches or five inches. The width can be, for example, 0.5 inch, 1 inch, 1.5 inches, 2 inches, or 2.5 inches. The cartridge can have a cross-section that is a circle, oval, or ellipse. In certain embodiments, opposing sides along the length of the cartridge can be flat. The thickness between the inner and outer surface can be constant, thereby providing a uniform exposure to the reducing agents. The thickness can be approximately 1 mm, 2 mm, 5 mm, 10 mm, 20 mm, 30 mm, or 40 mm for example.

Other features, objects, and advantages will be apparent from the description, drawings, and claims.

### BRIEF DESCRIPTION OF THE DRAWINGS

FIG. 1 is an illustration of a receptacle, which can be a cartridge.

FIGS. 2a) through c) are illustrations of a system including a receptacle.

FIG. 3 is a drawing depicting a system including a receptacle.

FIG. 4 is a graph showing nitric oxide and nitrogen dioxide concentrations as a function of time in comparison to a ventilator flow rate.

FIG. 5 is a graph showing nitric oxide and nitrogen dioxide concentrations as a function of time in comparison to a ventilator flow rate.

FIG. 6 is a graph showing nitric oxide concentration as a function of time in comparison to a ventilator flow rate.

FIG. 7 is a graph showing nitric oxide concentration as a function of time in comparison to a ventilator flow rate.

FIG. 8 is a graph showing nitric oxide concentration as a function of time in comparison to a ventilator flow rate.

FIG. 9 is a graph showing nitric oxide concentration as a function of time in comparison to a ventilator flow rate.

FIG. 10 is a schematic showing an embodiment of the claimed method.

FIG. 11 is a schematic showing an embodiment of the claimed method.

FIG. 12 is a schematic showing an embodiment of the claimed method.

FIG. 13 is a schematic showing an embodiment of the claimed method.

### DETAILED DESCRIPTION

Reactive oxygen species (ROS) are chemically reactive molecules containing oxygen. Examples include peroxides, superoxide, hydroxyl radical, and singlet oxygen. ROS are formed as a natural byproduct of the normal metabolism of oxygen and have important roles in cell signalling and homeostasis. However, during times of environmental stress (e.g., UV or heat exposure), ROS levels can increase dramatically. This may result in significant damage to cell structures. Cumulatively, this is known as oxidative stress. ROS are also generated by exogenous sources such as ionizing radiation. Clinicians can prescribe drugs for patients at risk for oxidative stress, and to manage fibrosis, for example. In addition, antioxidants are can be used in dietary supplements and have been investigated for the prevention of diseases such as cancer, coronary heart disease and altitude sickness.

Oxidative Stress

Oxidative stress reflects an imbalance between the systemic manifestation of reactive oxygen species and a biological system's ability to readily detoxify the reactive intermediates or to repair the resulting damage. Disturbances in the normal redox state of cells can cause toxic effects through the production of peroxides and free radicals that damage all components of the cell, including proteins, lipids, and DNA. Oxidative stress from oxidative metabolism causes base damage, as well as strand breaks in DNA. Base damage is mostly indirect and caused by reactive oxygen species (ROS) generated, e.g. O2—(superoxide radical), OH (hydroxyl radical) and H2O2 (hydrogen peroxide). Further, some reactive oxidative species act as cellular messengers in redox signalling. Thus, oxidative stress can cause disruptions in normal mechanisms of cellular signalling. Oxidative stress is suspected to be important in neurodegenerative diseases including Lou Gehrig's disease (aka MND or ALS), Parkinson's disease, Alzheimer's disease, Huntington's disease, and Multiple sclerosis. Indirect evidence via monitoring biomarkers such as reactive oxygen species, and reactive nitrogen species production, antioxidant defence indicates oxidative damage may be involved in the pathogenesis of these diseases, while cumulative oxidative stress with disrupted mitochondrial respiration and mitochondrial damage are related with Alzheimer's disease, Parkinson's disease, and other neurodegenerative diseases.

Oxidative stress is thought to be linked to certain cardiovascular disease, since oxidation of LDL in the vascular endothelium is a precursor to plaque formation. Oxidative stress also plays a role in the ischemic cascade due to oxygen reperfusion injury following hypoxia. This cascade includes both strokes and heart attacks. Oxidative stress has also been implicated in chronic fatigue syndrome. Oxidative stress also contributes to tissue injury following irradiation and hyperoxia, as well as in diabetes.

Oxidative stress is likely to be involved in age-related development of cancer. The reactive species produced in oxidative stress can cause direct damage to the DNA and are therefore mutagenic, and it may also suppress apoptosis and promote proliferation, invasiveness and metastasis.

Symptoms of oxidative stress include fatigue, memory loss and/or brain fog, muscle and/or joint pain, wrinkles and grey hair, decreased eye sight, headaches and sensitivity to noise, and susceptibility to infections. Oxidative stress can be reduced by avoiding exposure to unnecessary oxidation, increasing anti-oxidants, and by therapeutic treatment including administering pharmaceuticals.

Hemolysis

Hemolysis or the lysis of red blood cells is often associated with clot formation and hypertension. Identifying such a mammal having or at risk of developing a haemolytic condition typically includes making a diagnosis based on a physical examination including vital signs, laboratory tests (e.g. blood work, complete blood count (CBC), and metabolic panel including potassium and calcium levels) and ancillary testing (e.g., imaging studies for example). This typically further involves planning a course of treatment, communicating the diagnosis and treatment plan, and preparing the mammal for treatment. Exogenous nitric oxide treatment, such as inhaled NO, may be applied to mitigate clot formation and the hypertension related to hemolysis.

According to a 2008 study published in PLoS Medicine, an estimated 20,000 human deaths occur each year from snakebites, mostly in sub-Saharan Africa and Asia, though with the unreported incidents the total may be as high as 94,000 (animals.mom.me/snake-bite-death-statistics-worldwide-2431.html). Snake venoms can be neurotoxic, hemotoxic or a combination of both. The hemotoxic venoms can have combinations of proteolytic, hemorrhagic and hemolytic activity. Hemolytic toxins are also found in scorpion stings, sea anemones, and bacterial infections. In the case of hemolytic activity, the venom creates localized anemia and, though unlikely, secondary renal failure. Proteolysis results in coagulation of the blood in the blood vessels, which, in turn, damages red blood cells and becomes a secondary cause of hemolysis.

Remote Ischemic Conditioning

NO can be used alone or in combination with drugs (such as NO donors) to modulate the Remote Ischemic Conditioning pathway. For example, the administering of exogenous NO over a 30 minute period at low dose to cause accumulation of HIF (hypoxia inducible factor(s) and PHDs) to promote ROS signalling. This results in organ preservation by down regulating mitochondrial metabolic activity.

Identifying such a mammal having or at risk of developing an ischemic condition typically includes making a diagnosis based on a physical examination including vital signs, laboratory tests (e.g. blood work, complete blood count (CBC), and metabolic panel including potassium and calcium levels) and ancillary testing (e.g., imaging studies for example). This typically further involves planning a course of treatment, communicating the diagnosis and treatment plan, and preparing the mammal for treatment. Exogenous nitric oxide treatment, such as inhaled NO, may be applied to result in increased red blood cell (RBC) stimulation effect. Modulating HIF also results in erythropoietin production to stimulate red cell production. Thus, administering exogenous NO can be applied with other EPO stimulating drugs to manage, prevent and/or treat ischemic conditions.

ROS Reducing Drugs

ROS can trigger activation of signalling pathways involved in cell migration and invasion such as members of the mitogen activated protein kinase (MAPK) family—extracellular regulated kinase (ERK), c-jun NH-2 terminal kinase (JNK) and p38 MAPK. ROS can also promote migration by augmenting phosphorylation of the focal adhesion kinase (FAK) p130Cas and paxilin. Experimental and epidemiologic research over the past several years has indicated close associations among ROS, chronic inflammation, and cancer. ROS induces chronic inflammation by the induction of COX-2, inflammatory cytokines (TNFα, interleukin 1 (IL-1), IL-6), chemokines (IL-8, CXCR4) and pro-inflammatory transcription factors (NF-κB). These chemokines and chemokine receptors, in turn, promote invasion and metastasis of various tumor types.

Both in vitro and in vivo, ROS have been shown to induce transcription factors and modulate signalling molecules involved in angiogenesis (MMP, VEGF) and metastasis (upregulation of AP-1, CXCR4, AKT and downregulation of PTEN.

Cells have a variety of defence mechanisms that intercept free radicals to prevent or limit intracellular damage and ameliorate the harmful effects of ROS, including low-molecular-weight antioxidants (such as ascorbic acid, vitamin E, and glutathione) and antioxidant enzymes (such as thioredoxins, superoxide dismutase (SOD), catalase, and glutathione peroxidase). A key example of the latter is mitochondrial manganese superoxide dismutase (MnSOD), which converts superoxide radicals to hydrogen peroxide, which is further broken down into water by peroxidases. As a consequence of these activities, physiological levels of ROS are low. However, with heightened levels of ROS, defence systems can be overwhelmed resulting in cellular damage. Normally functioning cells can sustain and tolerate background levels of damage, but if an imbalance occurs, then cellular damage will increase. This damage may result from significant modification of intracellular targets such as DNA, proteins, and lipids and may modulate survival signalling cascades. See, e.g., Deavall, D., Drug-Induced Oxidative Stress and Toxicity, Journal of Toxicity, 2012 (Article ID 645460).

At the molecular level, the extent of damage depends on many factors including the site of ROS production, reactivity of the target, and the availability of metal ions. Modified proteins and lipids can be removed by normal cellular turnover, but DNA damage requires specific repair mechanisms. When mitochondrial DNA is the target of oxidation, it can lead to mutations, rearrangements, and transcriptional errors that impair important mitochondrial components, leading to more oxidative stress and eventual cell death. Molecular modifications in surviving cells can cause alterations in gene expression, and, depending on the severity and duration of ROS exposure, prosurvival or proapoptotic response pathways may be activated. Id.

Oxidative-stress-induced damage to DNA and macromolecules is associated with the onset and development of many diseases including cardiovascular disease, neurological degenerations (e.g., Alzheimer's disease, ischemic stroke), and cancer, as well as the normal ageing processes. Tumour cells have high levels of ROS, and studies have shown elevated levels of oxidative stress and/or oxidative DNA damage in human malignancies relative to normal cells. Generation of ROS at complex I of the electron transport chain (ETC), known as “complex I syndrome,” has been linked to age-associated modifications in the central nervous system. Conversely, the production of ROS and RNS is a key feature of some desirable immunological responses where, in response to activation by pathogens, phagocytes produce reactive species, including superoxide, nitric oxide, and peroxynitrite that can damage infected cells.

In addition to association with disease states, there is clear evidence to implicate drug-induced oxidative stress as a mechanism of toxicity in numerous tissues.

Anti-Fibrotic and Anti-Inflammatory Drugs

Certain drugs that inhibit TGF-β (e.g., Esbriet (pirfenidone)), and connective tissue growth factor (CTGF) reduce collagen production by myofibroblasts. These drugs have been identified and used to counteract the effects of reactive oxygen species and/or reduce fibrosis for both disease and drug-induced states. Therefore, exogenous NO can be administered alone or in combination with such drugs (e.g., pirfenidone) to inhibit TGF-B to inhibit fibrosis and the degree of inflammation.

Anti-Hypertensive Drugs

Antihypertensives are a class of drugs that are used to treat hypertension (high blood pressure). Antihypertensive therapy seeks to prevent the complications of high blood pressure, such as stroke and myocardial infarction. There are many classes of antihypertensives, which lower blood pressure by different means. Among the most important and most widely used drugs are thiazide diuretics, calcium channel blockers, ACE inhibitors, angiotensin II receptor antagonists (ARBs), and beta blockers.

Exogenous NO can also be administered alone or in combination with such drugs (e.g., nifedipine) to inhibit to treat, manage, or prevent hypertension.

TGF-β

Transforming growth factor beta (TGF-β) is a secreted protein that controls proliferation, cellular differentiation, and other functions in most cells, thereby acting to prevent inflammation and fibrosis. It is a type of cytokine which plays a role in immunity, cancer, bronchial asthma, lung fibrosis, heart disease, diabetes, hereditary hemorrhagic telangiectasia, Marfan syndrome, Vascular Ehlers-Danlos syndrome, Loeys-Dietz syndrome, Parkinson's disease, Chronic kidney disease, Multiple Sclerosis and AIDS.

TGF-β is secreted by many cell types, including macrophages, in a latent form in which it is complexed with two other polypeptides, latent TGF-beta binding protein (LTBP) and latency-associated peptide (LAP). Serum proteinases such as plasmin catalyze the release of active TGF-β from the complex. This often occurs on the surface of macrophages where the latent TGF-β complex is bound to CD36 via its ligand, thrombospondin-1 (TSP-1). Inflammatory stimuli that activate macrophages enhance the release of active TGF-β by promoting the activation of plasmin. Macrophages can also endocytose IgG-bound latent TGF-β complexes that are secreted by plasma cells and then release active TGF-β into the extracellular fluid.

TGF-β exists in at least three isoforms called TGF-β1, TGF-β2 and TGF-β3. Until the three isoforms were discovered, TGF-β referred to TGF-β1, as it was the first member of this family to be discovered. The TGF-β family is part of a superfamily of proteins known as the transforming growth factor beta superfamily, which includes inhibins, activin, anti-mullerian hormone, bone morphogenetic protein, decapentaplegic and Vg-1. Most tissues have high expression of the genes encoding TGF-β. In contrast, other anti-inflammatory cytokines such as IL-10 show minimal expression in unstimulated tissues and seem to require triggering by commensal or pathogenic flora. TGF-β acts as an antiproliferative factor in normal epithelial cells and at early stages of oncogenesis. Some cells that secrete TGF-β also have receptors for TGF-β. This is known as autocrine signalling. Cancerous cells increase their production of TGF-β, which also acts on surrounding cells.

Inhibition of TGF-B further minimizes recruitment of neutrophils and the associated inflammatory response.

CTGF

CTGF, also known as CCN2 or connective tissue growth factor, is a matricellular protein of the CCN family of extracellular matrix-associated heparin-binding proteins (see also CCN intercellular signalling protein). CTGF has important roles in many biological processes, including cell adhesion, migration, proliferation, angiogenesis, skeletal development, and tissue wound repair, and is critically involved in fibrotic disease and several forms of cancers.

CTGF is associated with wound healing and virtually all fibrotic pathology. It is thought that CTGF can cooperate with TGF-β to induce sustained fibrosis and to exacerbate extracellular matrix production in association other fibrosis-inducing conditions. Overexpression of CTGF in fibroblasts promotes fibrosis in the dermis, kidney, and lung, and deletion of Ctgf in fibroblasts and smooth muscle cells greatly reduces bleomycin-induced skin fibrosis.

In addition to fibrosis, aberrant CTGF expression is also associated with many types of malignancies, diabetic nephropathy and retinopathy, arthritis, and cardiovascular diseases. Several clinical trials are now ongoing that investigate the therapeutic value of targeting CTGF in fibrosis, diabetic nephropathy, and pancreatic cancer. TGF-B can directly or indirectly activate CTGF. Exogenous NO can be used to inhibit TGF-B, which in turn, decreases activation of CTGF and fibrosis. Therefore, exogenous NO can be administered alone or in combination with pirfenidone to inhibit TGF-B and CTGF to limit fibrosis.

IPF Drugs

Pulmonary fibrosis is a disease in which tissue deep inside the lungs becomes thick, stiff, and scarred, decreasing the lungs' ability to expand to take in air, and making it difficult to breathe. This is a progressive disease in which scarring and lack of elasticity in the lungs continues to increase until the patient can no longer breathe enough to sustain life.

Until recently, patients in the U.S. suffering from idiopathic pulmonary fibrosis (IPF), a form of pulmonary fibrosis in which the cause is unknown, had no drug treatment approved by FDA for this debilitating, incurable, and terminal condition. However, the FDA recently approved Ofev (nintedanib) and Esbriet (pirfenidone), two important new therapies for the treatment of patients with IPF. These drugs are believed to inhibit important pathways that help to prevent scarring. Neither drug is a cure, and IPF may still progress after patients use these drugs. However, each drug has been shown to significantly slow the progression of the disease.

Riociguat

Riociguat is a stimulator of soluble guanylate cyclase (sGC). Clinical trials have looked at riociguat as a new approach to treat two forms of pulmonary hypertension (PH): chronic thromboembolic pulmonary hypertension (CTEPH) and pulmonary arterial hypertension (PAH). Riociguat represents a class of sGC stimulators. Riociguat also activates soluble guanylyl cyclase with resultant vasodilation. The combination of Riociguat plus NO has been shown to potentiate the vasodilatory effect of the administered NO.

In healthy individuals nitric oxide (NO) acts as a signalling molecule on vascular smooth muscle cells to induce vasodilation. NO binds to soluble guanylate cyclase (sGC) and mediates the synthesis of the secondary messenger cyclic guanosine monophosphate (cGMP). sGC forms heterodimers consisting of a larger alpha-subunit and a smaller haem-binding beta-subunit. The synthesised cGMP acts as a secondary messenger and activates cGMP-dependent protein kinase (protein kinase G) to regulate cytosolic calcium ion concentration. This changes the actin-myosin contractility, which results in vasodilation. NO is produced by the enzyme endothelial nitric oxide synthetase (eNOS) NO synthase. In patients with pulmonary arterial hypertension eNOS levels are reduced. This results in overall lower levels of endothelial cell-derived NO and reduced vasodilation of smooth muscle cells. NO also reduces pulmonary smooth muscle cell growth and antagonises platelet inhibition, factors which play a key role in the pathogenesis of PAH. In contrast to NO- and haem-independent sGC activators like cinaciguat, the sGC stimulator riociguat directly stimulates sGC activity independent of NO and also acts in synergy with NO to produce anti-aggregatory, anti-proliferative, and vasodilatory effects. Grimminger F, Weimann G, Frey R, et al. (April 2009). “First acute haemodynamic study of soluble guanylate cyclase stimulator riociguat in pulmonary hypertension”. The European Respiratory Journal 33 (4): 785-92; Stasch J P, Hobbs A J (2009). “NO-independent, haem-dependent soluble guanylate cyclase stimulators”. Handbook of Experimental Pharmacology. Handbook of Experimental Pharmacology 191 (191): 277-308.

Therefore, the combination of administered NO with Riociguat may be used to gain a greater pulmonary hemodynamic effect reducing pressures and resistance compared to when each drug is used alone.

In short, riociguat potentiates the effect of NO. NO has an additive effect on the oral, inhaled and parenteral prostacyclin, PDGE-5 inhibitor (or similar drug) hemodynamic response. Administering NO in this manner with such drugs could extend the effective life of these other drugs and/or provide additional effectiveness during combined use.

Prostaglandins

Prostaglandins (PG) are a group of physiologically active lipid compounds having diverse hormone-like effects in animals. Prostaglandins have been found in almost every tissue in humans and other animals. They are derived enzymatically from fatty acids. Every prostaglandin contains 20 carbon atoms, including a 5-carbon ring. They are a subclass of eicosanoids and form the prostanoid class of fatty acid derivatives.

The structural differences between prostaglandins account for their different biological activities. A given prostaglandin may have different and even opposite effects in different tissues. The ability of the same prostaglandin to stimulate a reaction in one tissue and inhibit the same reaction in another tissue is determined by the type of receptor to which the prostaglandin binds. They act as autocrine or paracrine factors with their target cells present in the immediate vicinity of the site of their secretion. Prostaglandins differ from endocrine hormones in that they are not produced at a specific site but in many places throughout the human body.

Prostaglandins have two derivatives: prostacyclins and thromboxanes. Prostacyclins are powerful locally acting vasodilators and inhibit the aggregation of blood platelets. Through their role in vasodilation, prostacyclins are also involved in inflammation. They are synthesized in the walls of blood vessels and serve the physiological function of preventing needless clot formation, as well as regulating the contraction of smooth muscle tissue. Conversely, thromboxanes (produced by platelet cells) are vasoconstrictors and facilitate platelet aggregation. Their name comes from their role in clot formation (thrombosis).

The combination of protacyclins plus NO has been shown to potentiate the vasodilatory effect of the administered NO. Accordingly, the combination of administered NO with prostacyclins can similarly be used to gain a greater pulmonary hemodynamic effect reducing pressures and resistance compared to when each drug is used alone.

Caspases

NO can also be administered in combination with the drugs to target the Caspases. Caspases, the key effector molecules in apoptosis, together with a battery of triggers and regulators of their activity are among the most promising targets for pharmacological modulation of cell death. The search for caspase inhibitors was undertaken way before the discovery of these proteases as key-effectors in apoptosis. The target of interest has been the interleukin-1—-converting enzyme (ICE, now caspase-1). Caspase-1-4 and -5 are crucial regulators of secretion of inflammatory cytokines like IL-1B, IL-16, IL-18 and indirectly IFN-γ. In addition to caspases, modulators of their activity are also increasingly gaining the interest as potential targets for drug development. Among them the pro- and antiapoptotic Bcl-2 family members, especially the Bcl2 death inhibitor itself, are amid the most frequent targets. In recent years a family of caspase inhibitors called IAPs that bind and inactivate already active caspases attracted attention of the pharmaceutical industry. The interest in IAPs increased with the discovery of IAP inhibitors, Smac/DIABLO and HtrA2, that allow an additional level of apoptosis modulation. Depending on the part of IAP which would become occupied by a designed inhibitor, the net outcome could be either caspase activation and apoptosis if the interaction with caspase is disrupted, or downregulation of caspase activity and apoptosis inhibition, if the interaction with Smac/DIABLO becomes disrupted. Yet, another mechanism for apoptosis control can be applied. A number of cells express so called death receptors on the surface. They are able to activate caspases and induce apoptosis, when bound by appropriate ligand. A subfamily of caspases, termed apical/initiator caspases become activated upon enrollment to death inducing signalling complex (DISC), a multiprotein conglomerate recruited to death receptor within seconds, or minutes after its triggering. Once activated, the initiator caspases trigger downstream/effector caspases and other components of the apoptotic machinery. Modulation of interaction among DISC components, or triggering death receptors by naturally-occurring, or artificial ligand provides another mean of control of apoptotic process for clinical applications. Below we discuss in more details the progress, as well as positive and negative aspects of mentioned targets for drug development. See, e.g., Michalke, M., Caspases as Targets for Drug Development, Madame Curie Bioscience Database, Bookshelf ID: NBK6578, available at www.ncbi.nlm.nih.gov/books/NBK6578/?report=printable (last viewed Dec. 21, 2015).

ROS Reducing Agents

Cells such as cancer cells with elevated ROS levels depend heavily on the patient's antioxidant defense system. ROS-elevating drugs further increase cellular ROS stress level, either by direct ROS-generation (e.g. motexafin gadolinium, elesclomol) or by agents that abrogate the inherent antioxidant system such as SOD inhibitor (e.g. ATN-224, 2-methoxyestradiol) and GSH inhibitor (e.g. PEITC, buthionine sulfoximine (BSO)). The result is an overall increase in endogenous ROS, which when above a cellular tolerability threshold, may induce cell death. On the other hand, normal cells appear to have, under lower basal stress and reserve, a higher capacity to cope with additional ROS-generating insults than cancer cells do. While the elevation of ROS can achieve beneficial effects such as the selective killing of cancer cells, for example, such an effect must be modulated. ROS is a double-edged sword. On one hand, at low levels, ROS facilitates cancer cell survival since cell-cycle progression driven by growth factors and receptor tyrosine kinases (RTK) require ROS for activation and chronic inflammation, a major mediator of cancer, is regulated by ROS. On the other hand, a high level of ROS can suppress tumor growth through the sustained activation of cell-cycle inhibitor and induction of cell death as well as senescence by damaging macromolecules.

Cells control ROS levels by balancing the generation of ROS with their elimination by scavenging system. But under oxidative stress conditions, excessive ROS can damage cellular proteins, lipids and DNA, leading to fatal lesions in cell that contribute to carcinogenesis

ROS reducing drugs have been effective at modulating the adverse effects of ROS including the reduction of oxidative stress, and/or reducing fibrosis. Applicants have further discovered that the effects of TGF-B and CTGF can be modulated with administered NO to inhibit the actions of TGF-β and CTGF.

Combining NO with Supplemental Drug

Supplemental oxygen from a compressed tank or non-atmospheric source is a drug. The administration of NO according to the claimed methods, allows for a reduced oxygen requirement, and therefore allows a clinician to lower the dose of administered oxygen without compromising the effects of the administered oxygen. The result is the ability to minimize the symptoms and effects of oxidative stress and fibrosis.

Superoxide dismutases (SOD) are a class of enzymes that catalyze the dismutation of superoxide into oxygen and hydrogen peroxide. As such, they are an important antioxidant defense in nearly all cells exposed to oxygen. In mammals and most chordates, three forms of superoxide dismutase are present. SOD1 is located primarily in the cytoplasm, SOD2 in the mitochondria and SOD3 is extracellular. The first is a dimer (consists of two units), while the others are tetramers (four subunits). SOD1 and SOD3 contain copper and zinc ions, while SOD2 has a manganese ion in its reactive centre. The genes are located on chromosomes 21, 6, and 4, respectively (21q22.1, 6q25.3 and 4p15.3-p15.1).

Some disorders or physiological conditions that require supplemental oxygen can be mediated by inhalation of nitric oxide. The use of low concentrations of inhaled nitric oxide can prevent, reverse, or limit the progression of disorders which can include, but are not limited to, acute pulmonary vasoconstriction, traumatic injury, aspiration or inhalation injury, fat embolism in the lung, acidosis, inflammation of the lung, adult respiratory distress syndrome, acute pulmonary edema, acute mountain sickness, post cardiac surgery acute pulmonary hypertension, persistent pulmonary hypertension of a newborn, perinatal aspiration syndrome, haline membrane disease, acute pulmonary thromboembolism, heparin-protamine reactions, sepsis, asthma and status asthmaticus or hypoxia. Nitric oxide can also be used to treat chronic pulmonary hypertension, bronchopulmonary dysplasia, chronic pulmonary thromboembolism and idiopathic or primary pulmonary hypertension or chronic hypoxia, or conditions resulting from hemolysis or hemotoxic venoms. Advantageously, nitric oxide can be generated and delivered in the absence of harmful side products, such as nitrogen dioxide. The nitric oxide can be generated at a concentration suitable for delivery to a mammal in need of treatment such that supplemental oxygen is administered to achieve a target effect while minimizing oxidative damage to a patient's tissues.

When delivering nitric oxide (NO) for therapeutic use to a mammal, it is also important to avoid delivery of nitrogen dioxide (NO₂) to the mammal. Nitrogen dioxide (NO₂) can be formed by the oxidation of nitric oxide (NO) with oxygen (O₂). The rate of formation of nitrogen dioxide (NO₂) can be proportional to the oxygen (O₂) concentration multiplied by the square of the nitric oxide (NO) concentration. A NO delivery system can convert nitrogen dioxide (NO₂) to nitric oxide (NO). Additionally, nitric oxide can form nitrogen dioxide at increased concentrations.

Additional Indications for NO with Supplemental Drugs

INO targets soluble guanylate cyclase . This enzyme mediates many of the biological effects of NO and is responsible for the conversion of GTP to cGMP. Therefore, NO can be used to modulate the guanylate cyclase-13 GTP-cGMP pathways alone or in combination with other drugs identified below to control the pathological biochemical pathways. Targets that have been identified are the basis for treatment options to manage oxygen species (OS) and the profibrotic pathways that could be used alone or in combination are as described below. These targets could be used in combination with NO, or independently, or new indications for use.

1. Nitric Oxide Synthase Pathway
Over production of endogenous NO via the nitric oxide synthase pathway may result in an explosive reaction between the NO that is generated and superoxides leading to the production of peroxynitrite. Use of our method and apparatus to deliver exogenous NO alone or in combination with other agents such as those listed below may suppress endogenous NO production via the NO synthase pathway minimizing the reaction with superoxides and the formation of peroxynitrite.

2. Superoxide Dismutase—Provides Protection by Limiting Superoxide Formation
Superoxide dismutase (SOD) normally functions to minimize OS by limiting the amount of superoxides that are formed. SOD may be decreased under OS conditions rendering the cell more vulnerable to superoxide formation which can react with endogenous NO and form peroxynitrite. Another strategy to be considered in managing OS is to implement methods alone or in combination with our NO delivery system to: a) boost the SOD levels for patients at risk of oxidative damage, and b) suppress endogenous NO production. One agent that has been explored to increase the amount of SOD is Nifedipine, a calcium antagonist. Fukuo found that nifedipine indirectly upregulates endothelial SOD expression by stimulating vascular endothelial growth factor (VEGF) production from adjacent vascular smooth muscle cells. IP targeting methods that increase SOD can be pursued alone or in combination with one or more of the other OS targets/methods described for example in Kelly, G S, Alternative Medicine Review: a Journal of Clinical Therapeutic [1998, 3(2): 114-127], which is incorporated by reference herein.

3. Lipid Peroxidases—May Cause Permanent Damage to Cell Membranes and Cell Death
A key event in OS is lipid peroxidation resulting in oxidative degeneration of lipids. This is caused by a free radical chain reaction affecting membrane polyunsaturated fatty acids. Failure to control lipid peroxidation can lead to triggering secondary messengers, cell signalling and DNA damage. An end product of lipid peroxidation is hydroxyl-2-nonenal (HNE) which may potentiate OS through depletion of glutathione. HNE also plays a role in airway remodelling by activation of epidermal growth factor and induction of fibronectin production. Therefore, inhibition of OS/fibrosis may be controlled by the pathways described above with our delivery system in combination with inhibition of the lipid peroxidase pathways using agents to inhibit hydroxyl-2-nonenal (HNE) along with other potential targets.

4. Glutathione—Patients at Risk of OS-Related Damage Are Glutathione Deficient
Glutathione is a potent antioxidant that serves to protect the body against OS by minimizing peroxynitrite. Methods described below that increase glutathione may enhance protection against OS-related damage. N-acetylcysteine (NAC), the acetylated variant of the amino acid L-cysteine, is an excellent source of sulfhydryl (SH) groups, and is converted in the body into metabolites capable of stimulating glutathione (GSH) synthesis, promoting detoxification, and acting directly as free radical scavengers. IP targeting methods that increase NAC, and therefore glutathione, can be pursued alone or in combination with one or more of the other OS/fibrosis targets & methods described in Kelly, G S, Alternative Medicine Review: a Journal of Clinical Therapeutic [1998, 3(2): 114-127], which is incorporated by reference herein.

5. Use of Negative Inotropes—Reduce Contractility, Oxygen Consumption & OS
Administration of low-dose negative inotropes including endothelin inhibitors early in the course of disease may retard or inhibit the progression of the disease by managing OS. The negative inotropes may reduce contractility, oxygen consumption and mitochondrial activity. A reduction in the metabolic activity is anticipated to reduce the amount of superoxides and peroxynitrite formed. Peroxynitrite has been shown to lead to fibrosis in both IPF and HF. Reducing peroxynitrite formation early in the disease may limit fibrosis and other sequelae related to OS.

6. Potential Drug Targets to Control TGF-B 1:

OS has been shown to lead to an increase in TGF-B1 with resulting fibrosis. The following targets have been identified that may reduce the formation of TGF-B 1
1. Protease inhibitors of the TGF-β31 activation. Inhibitors could target MMP2 and MMP9.
2. ACE inhibitors
3. Active treatment of OS to mitigate amplification of the TGF-β31 cycle
4. Increase NO without the presence of superoxide
5. Method and apparatus to deliver iNO in combination with Pirfenidone, a TGF-B blocker
6. Inhibition for TGF-B activation of Connective Tissue Growth Factor
7. A combination of the above.

7. Drugs that Directly or Indirectly Target Connective Tissue Growth Factor (CTGF)

Connective tissue growth factor (CTGF), a profibrotic cytokine, acts downstream and in concert with TGF-β to stimulate the fibrotic process and is involved in the fibrosis seen in scleroderma. Iloprost, acting by elevation of cAMP, blocks the induction of CTGF and the increase in collagen synthesis in fibroblasts exposed to TGF-β. its effect is mediated by the prostacyclin receptor IP. CTGF levels are greatly elevated in the dermis of scleroderma patients compared with healthy controls and Iloprost infusion causes a marked decrease in dermal CTGF levels. Iloprost could reduce the level of a key profibrotic cytokine in scleroderma patients and endogenous production of eicosanoids may limit the fibrotic response to TGF-β. See, e.g., J. Clin Invest. 2001;108(2):241-250, incorporated by reference herein.
PPARγ inhibits TGF-β-induced CTGF expression by directly interfering with the Smad3 signaling pathway. See, e.g., Fu, et al., Peroxisome Proliferator-activated Receptor Inhibits Transforming Growth Factor y-induced Connective Tissue Growth Factor Expression in Human Aortic Smooth Muscle Cells by Interfering with Smad3 J. Biol. Chem. 2001, 276 (49):45888-45894, incorporated by reference herein.
Connective tissue growth factor (CTGF), a potent profibrotic mediator, acts downstream and in concert with transforming growth factor (TGF)-β to drive fibrogenesis. Significant up-regulation of CTGF has been reported in fibrogenic diseases, including idiopathic pulmonary fibrosis (IPF), and is partly responsible for associated excessive fibroblast proliferation and extracellular matrix deposition. Simvastatin has reported putative antifibrotic actions in renal fibroblasts. Simvastatin reduces basal CTGF gene and protein expression in all fibroblast lines, overriding TGF-β induction through inhibition of the cholesterol synthesis pathway. See, e.g., Watts, Connective tissue growth factor expression and induction by transforming growth factor-β is abrogated by simvastatin via a Rho signalling mechanism. American J. of Physiology-Lung Cellular and Molecular Physiology, December 2004 Vol. 287 no. 6, L1323-L1332, incorporated by reference herein.

8. Drugs that Target Platelet Derived Growth Factor (PDGF)
PDGFs drive pathological mesenchymal responses in vascular disorders such as atherosclerosis, restenosis, pulmonary hypertension, and retinal diseases, as well as in fibrotic diseases, including pulmonary fibrosis, liver cirrhosis, scleroderma, glomerulosclerosis, and cardiac fibrosis. Paracrine PDGF signaling may be involved in epithelial-mesenchymal transition.
One of the most efficient ways to block PDGFR (platelet derived growth factor receptor) signaling is to inhibit the PDGFR kinase activity. Kinase inhibitors act by binding at or near the ATP-binding pocket of the kinase domain. Several kinase inhibitors have been developed that block PDGFRs, but the inhibitors available so far are not completely specific. One of them, imatinib mesylate (Gleevec), inhibits PDGFR-α and PDGFR-β. Andrae, et al., Role of platelet-derived growth factors in physiology and medicine. Genes Dev. 2008 May 15; 22(10): 1276-1312. doi: 10.1101/gad.1653708.
Therefore, inhibition of OS/fibrosis may be controlled by the pathways described above with our delivery system in combination with Imatinib mesylate (Gleevec) which inhibits PDGFR-α and PDGFR-β along with other potential targets.

9. Drugs that Target NOX4-(NADPH oxidase 4 (Nox4)

Targeting Nox4 with GKT137831 provides a novel strategy to attenuate hypoxia-induced alterations in pulmonary vascular wall cells that contribute to vascular remodelling and RVH, key features involved in PH pathogenesis. Nox4 plays an important role in the pathophysiology of a wide variety of disorders, including systemic hypertension, diabetes mellitus, vascular injury, atherosclerosis, ischemic stroke, pulmonary fibrosis, and diabetic nephropathy. Nox4 oxidase is a major contributor to oxidative stress in these pathologic conditions, and blocking the undesirable actions of Nox4 could become a therapeutic strategy to attenuate oxidative stress in patients with these disorders. See, e.g, Am J Respir Cell Mol Biol. 2012 Nov;47(5):718-26. doi: 10.1165/rcmb.2011-04180C. Epub 2012 Aug 16. The Nox4 inhibitor GKT137831 attenuates hypoxia-induced pulmonary vascular cell proliferation. Green D E, Murphy T C, Kang B Y, Kleinhenz J M, Szyndralewiez C, Page P, Sutliff R L, Hart C M. International Journal of Hypertension Volume 2013 (2013), Article ID 842827, 9 pages Strategies Aimed at Nox4 Oxidase Inhibition Employing Peptides from Nox4 B-Loop and C-Terminus and p22^{phox} N-Terminus: An Elusive Target. Gábor Csányi and Patrick J. Pagano, incorporated by reference herein.

10. Drugs that Inhibit Chemotaxis Leading to Neutrophil, Macrophage, Mast Cell Infiltration

Azole derivatives exert direct anti-inflammatory activity. Inhibition of PMN (Neutrophil) chemotaxis and leukotriene biosynthesis has been suggested to explain this phenomenon. NAD+ derived metabolites regulate PMN chemotaxis by inducing extracellular Ca2+ influx via the ion channel TRPM2. Econazole and clotrimazole significantly inhibited PMN directionality. Econazole, clotrimazole as well as FA, another compound proposed to block the TRPM2 channel, blocked Ca2+ influx rendering defective Ca2+ responses comparable to those responses observed in fMLP-stimulated TRPM2 KO PMN. Compounds which antagonize TRPM2-mediated Ca2+ signalling pathway may function as effective inhibitors of PMN recruitment.

Therefore, inhibition of OS/fibrosis may be controlled by the pathways described above with our delivery system in combination with Azole derivatives as described above to prevent neutrophil recruitment along with other potential targets. See, e.g., Santiago Partida-Sanchez, Adriana Sumoza-Toledo, Harivadan Bhagat, Ingo Lange, Hanna Cortado, and Andrea Fleig, Azole derivative drugs inhibit neutrophil chemotaxis by blocking the Calcium permeant channel TRPM2. The Journal of Immunology, 2010, 184, incorporated by reference herein.

11. Drugs that Reduce Metabolic and Mitochondrial Activity to Decrease Production of Superoxides

Approximately 1-3% of oxygen utilized in the mitochondria is in the form of superoxides. Increased metabolic activity may lead to an increase in the production of superoxides. Agents that minimize an increase in mitochondrial metabolism may reduce superoxide production, and therefore, the risk of oxidative stress.

12. Inhibition of Type IV Collagenase Activity

Myofibroblasts break down the alveolar epithelial basement membrane during the profibrotic process. The basement membrane is comprised of Type IV Collagen. The myofibroblasts produce collagenases that catabolize the collagen. Hence, the use of collagenase inhibitors may protect the basement membrane and inhibit the progression of fibrosis and possibly epithelial mesenchymal transition. Inhibition of Type IV collagenases may be accomplished through the use of a novel cyclic peptide inhibitor CTTHWGFTLC (CTT) for matrix metalloproteinases (MMP)-2 and MMP-9, two types of Type IV collagenases or gelatinases.

13. Angiotensin Converting Enzyme (ACE) Inhibitors and Inhibition of Angiotensin II(AT-II) Assist in Preventing Fibrosis

ACE inhibitors have been shown to reduce fibrosis by reducing OS. AT II and TGF-β both activate the Smad protein system, which leads to the expression of genes related to fibrosis. In fibrotic conditions, such as tubulointerstitial nephritis, systemic sclerosis, and myocardial infarctions, AT II acts both independently and synergistically with TGF-β. Both AT II and TGF-β act through a messenger system, the Smad proteins that lead to excessive extracellular matrix formation.

Angiotensin II (A II) is a pro-oxidant and fibrogenic cytokine. Ang II stimulates DNA synthesis, cell migration, pro-collagen α1(I) mRNA expression, and secretion of TGF-β1 and inflammatory cytokines. These effects are attenuated by N-acetylcysteine and diphenylene iodonium, an NADPH oxidase inhibitor. NADPH oxidase mediates the actions of A II and plays a critical role in liver fibrogenesis. Bataller, et al. NADPH oxidase signal transduces angiotensin II in hepatic stellate cells and is critical in hepatic fibrosis, J Clin Invest. 2003;112(9):1383-1394. doi:10.1172/JCI18212.

Treatment Options

There are several novel options for managing oxidative stress that include, but are not limited to:

A method and apparatus for iNO delivery in combination with agents such as calcium antagonists (eg. Nefedipine) to induce upregulation of superoxide dismutase (SOD) to reduce the formation of superoxides and the risk of OS.

Administration of agents to upregulate deficient glutathione levels to reduce oxidative stress

A method and apparatus for minimizing the formation of NO2 to inhibit lipid peroxidase production to reduce OS

A method and apparatus to slowly reduce iNO levels during weaning in combination with use of systemic vasodilators such as sildenafil (phosphodieasterase-5 inhibitors) during the weaning process from NO and afterwards to prevent rebound

A method and apparatus to slowly reduce iNO levels during weaning in combination with inhaled or oral endothelin inhibitors to prevent rebound.

The method can also include Inhibition of OS and/or fibrosis by controlling the pathways described above using: a) an NO delivery system alone, b) an NO delivery system in combination with one or more of the drugs listed below, or c) a single drug listed below using a novel dose or in combination with other drugs.

This treatment can achieve inhibition of platelet derived growth factor (PDGF) using azole derivatives (as described above) to prevent neutrophil recruitment and the inflammatory response. Example: Gleevec (Iimatinib mesylate) which inhibits PDGFR-α and PDGFR-β (Platelet derived growth factor receptors alpha and beta);

The treatment can also achieve inhibition of the lipid peroxidase pathways using agents to inhibit hydroxyl-2-nonenal (HNE);

The treatment can also achieve stimulation of glutathione production with agents such as N-Acetylcysteine (NAC), a synthetic precursor of intracellular cysteine and glutathione, to minimize peroxynitrite and OS. Increasing the cell content of CoA by supplying pantothenic acid, can also be used as to boost glutathione levels. Glutathione content and its reduction state can also be increased by incubating the cells with curcumin, the yellow pigment of the Indian spice curry or with the analgesic drug flupirtine. Other compounds acting as general intracellular antioxidants are ascorbic acid (vitamin C), α-tocopherol (vitamin E), β-carotene, and α-lipoic acid. All these compounds are naturally present in the cell, but their contents can be increased when they are additionally administered. See, e.g., Szewczyk, A., Mitochondria as a Pharmacological Target, Pharmacological Reviews Mar. 1, 2002 vol. 54 no. 1 101-127.

The treatment can also achieve inhibition of TGF-□1 activation and fibrosis through the use of one or more of the following: (a) Protease inhibitors that could target MMP2 and MMP9; (b) ACE inhibitors; (c) Inhaled NO to minimize activation of the OS pathway; (d) iNO in combination with Pirfenidone, a TGF-B blocker; or (d) a combination of any or all of the above.

The treatment can also achieve inhibition for TGF-B activation using the techniques above to inhibit production of Connective Tissue Growth Factor.

The treatment can also achieve inhibition of Connective Tissue Growth Factor with agents such as Iloprost. Iloprost acts by elevating cAMP. (cAMP blocks the induction of CTGF and the increase in collagen synthesis in fibroblasts exposed to TGF-β. Its effect is mediated by the prostacyclin receptor IP).

The treatment can also achieve inhibition of Nox4 with GKT137831. The treatment can also be a method and apparatus to deliver nitric oxide for the purpose of inhibiting the TGF-b/NADH oxidase (NOX)/H2O2/Fenton reaction/Fibrosis pathway.

The treatment can also achieve inhibition of mitochondrial metabolic activity to reduce ROS/OS by reducing supplemental oxygen and through the use negative inotropes or local anesthetics. An example of negative inotrope is B-Blockers. Additional negative inotropes are listed in the Table below. Local anesthetics comprised of tertiary amines affect mitochondrial energy metabolism by uncoupling oxidative phosphorylation and inhibiting mitochondrial ATPase. Bupivacaine also uncouples oxidative phosphorylation. Dibucaine promotes the inhibition of a Ca2+ -induced increase in mitochondrial ROS generation.

The treatment can also achieve inhibition of Type IV Collagenase activity to minimize disruption of the basement membrane (for example associated with alveolar epithelial cells). Examples include the use of the novel cyclic peptide inhibitor CTTHWGFTLC (CTT) for matrix metalloproteinases (MMP)-2 and MMP-9, two types of Type IV collagenases or gelatinases. Augmented killing of cells was obtained by the CTT-enhanced delivery of Adriamycin-containing liposomes, compared with control liposomes administered without the peptide See, e.g, http://cancerres.aacrjournals.org/content/61/10/3978. short.

The treatment can also achieve inhibition of ACE and Angiotensin II (AT-II) to prevent fibrosis. AT II and TGF-β both activate the Smad protein system, which leads to the expression of genes related to fibrosis. A-II can be inhibited through the use of N-acetylcysteine and diphenylene iodonium, an NADPH oxidase inhibitor. See, e.g., http://www.jci.org/articles/view/18212.

The treatment can also minimize fibrosis by administering platelet factor antagonists such as BN 52021.

In sum, generally, the administration of antioxidants will increase total antioxidant activity in the cell to minimize OS in a patient with elevated ROS.

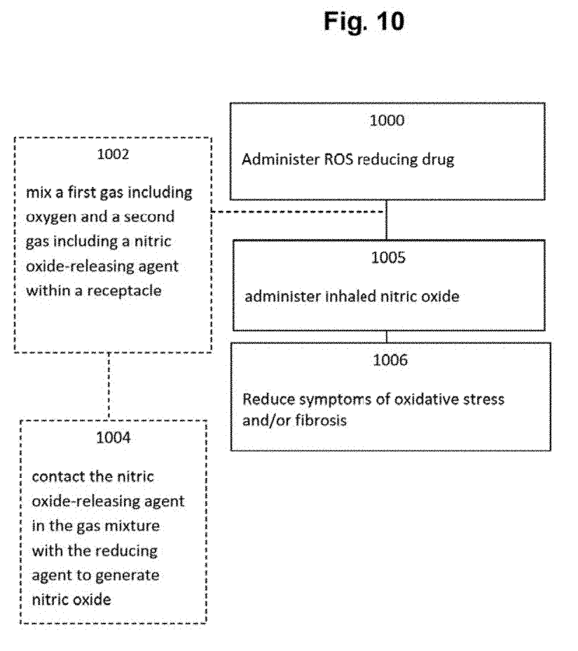

Referring to FIG. 10, a method of providing a therapeutic composition includes administering a ROS reducing drug 1000, administering an inhaled nitric oxide 1005 and reducing symptoms of oxidative stress and/or fibrosis in a patient 1006. The method can also include mixing a first gas including oxygen and a second gas including a nitric oxide-releasing agent within a receptacle to form a gas mixture 1002, wherein the receptacle includes an inlet, an outlet and a reducing agent and contacting the nitric oxide-releasing agent in the gas mixture with the reducing agent to generate nitric oxide 1004. Steps 1002 and 1004 are optional, and are preferably performed before administering the inhaled nitric oxide.

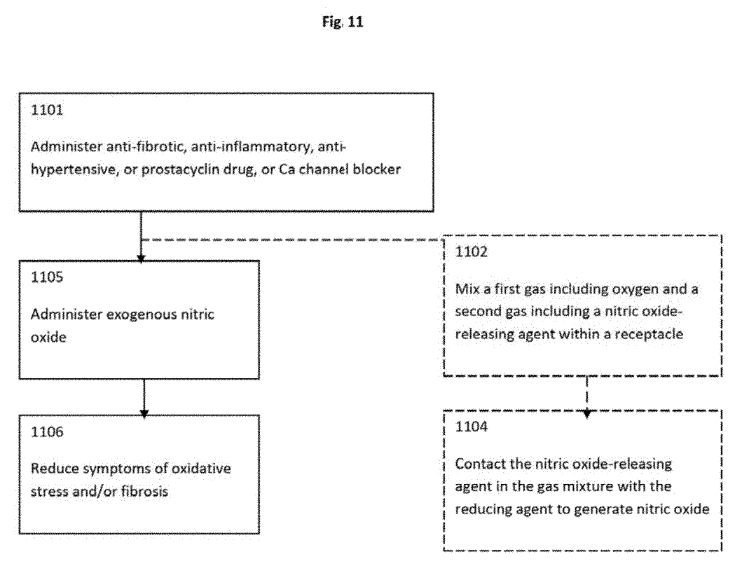

Referring to FIG. 11, a method of providing a therapeutic composition includes administering an anti-fibrotic, anti-inflammatory, anti-hypertensive, or prostacyclin drug, or Ca channel blocker 1101, administering exogenous nitric oxide 1105 and reducing symptoms of oxidative stress and/or fibrosis in a patient 1106. The method can also include mixing a first gas including oxygen and a second gas including a nitric oxide-releasing agent within a receptacle to form a gas mixture 1102, wherein the receptacle includes an inlet, an outlet and a reducing agent and contacting the nitric oxide-releasing agent in the gas mixture with the reducing agent to generate nitric oxide 1104. Steps 1102 and 1104 are optional, and are preferably performed before administering the inhaled nitric oxide.

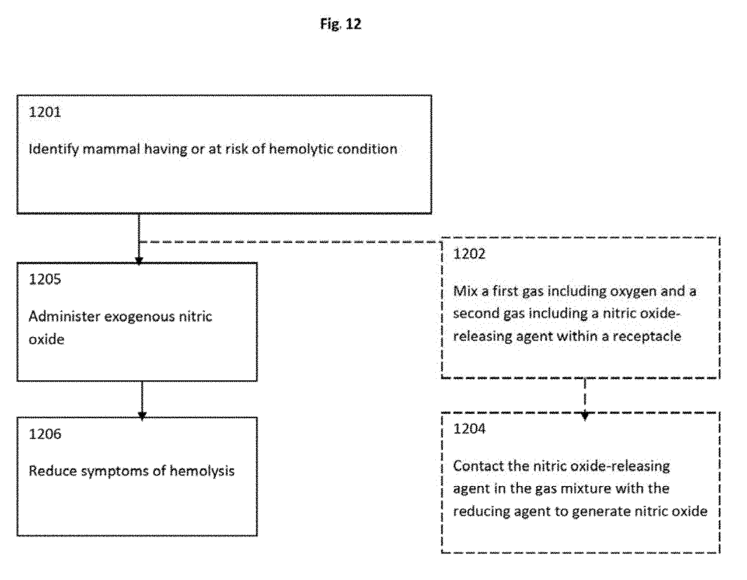

Referring to FIG. 12, a method of providing a therapeutic composition includes identifying a mammal having or at risk of developing a hemolytic condition (such as a venom from a snake bite) 1201, administering exogenous nitric oxide 1205 and reducing symptoms of hemolysis in the mammal such as a patient 1206. The method can also include mixing a first gas including oxygen and a second gas including a nitric oxide-releasing agent within a receptacle to form a gas mixture 1202, wherein the receptacle includes an inlet, an outlet and a reducing agent and contacting the nitric oxide-releasing agent in the gas mixture with the reducing agent to generate nitric oxide 1204. Steps 1202 and 1204 are optional, and are preferably performed before administering the inhaled nitric oxide.

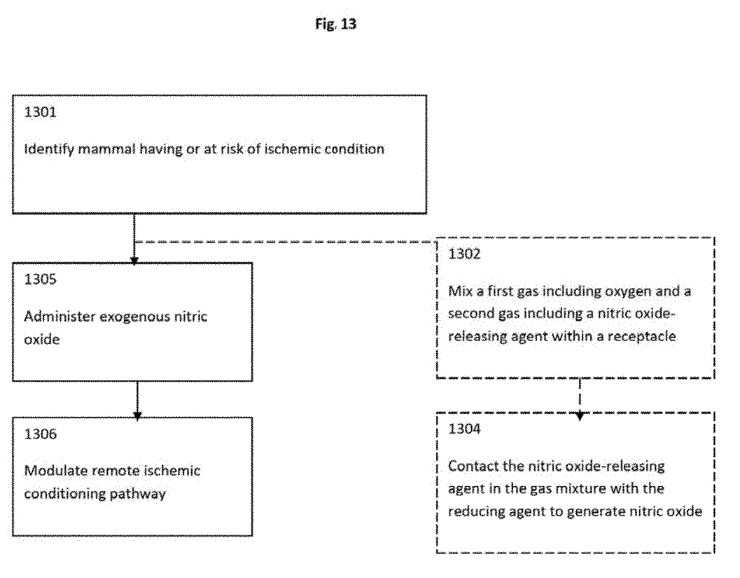

Referring to FIG. 13, a method of providing a therapeutic composition can include identifying a mammal having or at risk of developing an ischemic condition 1301, administering exogenous nitric oxide 1305; and administering a drug with the nitric oxide to modulate remote ischemic conditioning pathway 1306. In certain embodiments, the exogenous NO can be administered over a 30 minute period at low dose effective to cause accumulation of hypoxia inducible factor(s) and PHDs to promote ROS signalling. In other embodiments, the method can improve organ preservation by down regulating mitochondrial metabolic activity.

The method can also include mixing a first gas including oxygen and a second gas including a nitric oxide-releasing agent within a receptacle to form a gas mixture 1302, wherein the receptacle includes an inlet, an outlet and a reducing agent and contacting the nitric oxide-releasing agent in the gas mixture with the reducing agent to generate nitric oxide 1304. Steps 1302 and 1304 are optional, and are preferably performed before administering the inhaled nitric oxide.

A method of modulating oxygen saturation levels can include measuring oxygen saturation levels in a patient administering inhaled nitric oxide, adjusting the dose of oxygen in real time to a second dose based on the inhaled nitric oxide determining a first oxygen requirement to address an oxygen deficiency, determining a reduced oxygen requirement based on the generated nitric oxide, and delivering a dose of supplemental oxygen based on the reduced oxygen requirement and the gas mixture including nitric oxide from the receptacle to the patient. Adjusting the dose includes titrating the dose of oxygen in real time.

The method can also include mixing a first gas including oxygen and a second gas including a nitric oxide-releasing agent within a receptacle to form a gas mixture, wherein the receptacle includes an inlet, an outlet and a reducing agent and contacting the nitric oxide-releasing agent in the gas mixture with the reducing agent to generate nitric oxide.

The method of modulating oxygen saturation levels can also include measuring oxygen saturation levels in a patient, determining a first dose of oxygen to address an oxygen deficiency, mixing a first gas including oxygen and a second gas including a nitric oxide, determining a second dose of oxygen based on an amount of nitric oxide to be co-administered with the oxygen, wherein the second dose is lower than the first dose; and delivering the gas mixture including nitric oxide from the receptacle to the patient.

Situations Requiring Supplemental Oxygen

The administration of supplemental oxygen is an essential element of appropriate management for a wide range of clinical conditions, spanning different medical and surgical specialities. In general, the clinical goals of oxygen therapy are to treat hypoxemia, decrease the work of breathing and/or decrease myocardial work. The most common reasons for oxygen therapy to be initiated include acute hypoxemia such as that caused by shock, asthma, pneumonia or heart failure, ischemia such as cause by myocardial infarction, an abnormality in the quality or type of haemoglobin, acute blood loss in trauma or cyanide poisoning. A patient's need for oxygen therapy is based on a specific clinical condition. Oxygen therapy is prescribed for patients unable to get enough oxygen independently, often because of a lung condition that prevents the lings from absorbing oxygen, including COPD, pneumonia, asthma, dysplasia (or underdeveloped lungs in newborns), heart failures, cystic fibrosis, sleep apnea, lung disease, or trauma to the respiratory system.

Oxygen therapy is prescribed for both acute (short term) and chronic (long term) conditions and diseases. Short-term oxygen is usually prescribed for severe pneumonia, several asthma, respiratory distress syndrome (RDS) or bronchopulmonary dysplasia (BPD) in premature babies. Pneumonia involves an infection that causes a lung's air sacs to become inflamed. This prevents the air sacs from moving enough oxygen to the blood. In a severe asthma attack, the airways become inflamed and narrowed. While most people with asthma can manage their symptoms, a severe asthma attack can require hospitalization and oxygen therapy. Finally, premature babies may receive extra oxygen through a nasal continuous positive airway pressure (NCPAP) machine or a ventilator, or through a nasal tube.

Long-term oxygen therapy can be used for certain conditions such as chronic obstructive pulmonary disease (COPD), pulmonary fibrosis, cystic fibrosis (CF), emphysema, chronic bronchitis, alpha 1 antitrypsin deficiency, and sleep-related breathing disorders. COPD is a progressive disease in which damage to the air sacs prevents them from moving enough oxygen into the bloodstream. “Progressive” means the disease gets worse over time.

CF is an inherited disease of the secretory glands, including the glands that make mucus and sweat. People who have CF have thick, sticky mucus that collects in their airways. The mucus makes it easy for bacteria to grow. This leads to repeated, serious lung infections. Over time, these infections can severely damage the lungs.

Emphysema is diagnosed when the small air sacs in the lungs gradually become compromised and the damage makes it harder to breathe normally. Those with emphysema often become short of breath on a regular basis. However, supplemental oxygen can help provide some relief by increasing blood oxygen levels and making oxygen distribution easier on the body.

Chronic bronchitis can also be caused by cigarette smoke and harmful toxins and pollutants breathed in over time. The disease, which will get worse over time, is characterized by a constant cough and large amount of mucus. When caught early, the disease can then be managed.

Alpha 1 antitrypsin deficiency is a genetic disorder that can lead to breathing problems at a young age and eventually develop into emphysema or Chronic Obstructive Pulmonary Disease (COPD). The Alpha 1 Antitrypsin enzyme is found in the lungs and bloodstream and is meant to prevent inflammation and its effects in the lungs. When a patient's body lacks enough of this enzyme, it can lead to emphysema and make it difficult to breathe. Supplemental oxygen, along with bronchodilators and pulmonary rehabilitation, are common treatments.

Sleep-related breathing disorders that lead to low levels of oxygen in the blood during sleep, such as sleep apnea and late stage heart failure can also require oxygen therapy. This is a condition in which the heart is unable to pump enough oxygen-rich blood to meet the body's needs.

Measuring Oxygen Saturation Levels

In patients in need of oxygen therapy, the first step is to measure the patient's oxygen saturation levels. This measurement is typically conducted using pulse oximetry. A pulse oximeter is a medical device that indirectly monitors the oxygen saturation of a patient's blood (as opposed to measuring oxygen saturation directly through a blood sample) and changes in blood volume in the skin. The pulse oximeter may be incorporated into a multi-parameter patient monitor. Most monitors also display the pulse rate. Portable, battery-operated pulse oximeters are also available for transport or home blood-oxygen monitoring.

In pulse oximetry, a transdermal sensor is placed on a thin part of the patient's body such as a fingertip or earlobe, or in the case of an infant, across a foot. The device passes two wavelengths of light through the body part to a photodetector. The photodetector measures the changing absorbance at each of the wavelengths, allowing it to determine the absorbances due to the pulsing arterial blood. Pulse oximetry is available for certain smartphones.

Alternatively, reflectance pulse oximetry may be used, which does not require selecting a thin section of the person's body and is therefore well suited to more universal applications, such as the feet, forehead and chest. However, this method also has so limitations. Vasodilation and pooling of venous blood in the head due to compromised venous return to the heart, as occurs with congenital cyanotic heart disease patients, or in patients in the Trendelenburg position, can cause a combination of arterial and venous pulsations in the forehead region and lead to spurious SpO2 (Saturation of peripheral oxygen) results.

Determining Supplemental Oxygen Requirement

Based on the measured oxygen saturation levels and the diagnosis of the patient's condition, a medical provider such as a physician then determines and selects an effective dose of supplemental oxygen to administer to a patient. A healthy patient's baseline oxygen saturation levels are typically 98-100 percent. If a patient's oxygen saturation levels are below 90 percent, supplemental oxygen therapy is usually required, and the appropriate dose of supplemental oxygen is determined based on the deficiency. For example, if the measure oxygen saturation level is 80 percent, a typical dose of supplemental oxygen for low flow delivery devices is 1-6 L/min. via nasal cannula and 5-6 L/min via oxygen mask. High flow delivery devices can offer a typical dose of about 30 L/min, or higher.

Depending on the diagnosed condition, the goal of supplemental oxygen is generally to maintain a PaO₂ of 55-60 mmHg, which corresponds to SpO₂ of about 90%. Higher concentrations of oxygen can blunt the hypoxic ventilatory drive, which may precipitate hypoventilation and CO₂ retention.

The fraction of inspired oxygen (FiO₂) is the fraction or percentage of oxygen in the space being measured. Medical patients experiencing difficulty breathing are provided with oxygen-enriched air, which means a higher-than-atmospheric FiO₂. Natural air includes 20.9% oxygen, which is equivalent to FiO₂ of 0.209. Oxygen-enriched air has a higher FiO₂ than 0.21, up to 1.00, which means 100% oxygen. FiO₂ is typically maintained below 0.5 even with mechanical ventilation, to avoid oxygen toxicity. If a patient is wearing a nasal cannula or a simple face mask, each additional liter of oxygen adds about 4% to their FiO₂ (for example, a patient with a nasal cannula with 2L of oxygen attached would have an FiO₂ of 21%+8%=29%). The ratio of partial pressure arterial oxygen and fraction of inspired oxygen, sometimes called the Carrico index, is a comparison between the oxygen level in the blood and the oxygen concentration that is breathed.

Potential Adverse Effects of Oxygen

In general, oxygen therapy is safe and effective. The net effect of oxygen therapy is to reverse hypoxaemia and the benefits generally outweigh the risks. However, hazards of oxygen therapy that a clinician must recognize include oxygen toxicity and CO₂ retention. While there is a growing acknowledgement of oxygen as a drug with specific biochemical and physiologic actions in a distinct range of effective doses, there are also well-defined adverse effects at high doses.

Patients exposed to inspiratory oxygen fraction (FiO₂)>50% may experience oxygen toxicity, particularly if the exposure is prolonged. Oxygen toxicity is related to free radicals. The major end product of normal oxygen metabolism is water. Some oxygen molecules, however, are converted into highly reactive radicals, which include superoxide anions, perhydroxy radicals and hydroxyl radicals, and are toxic to alveolar and tracheobronchial cells.

Pathophysiological changes include decreased lung compliance, reduced inspiratory airflow, decreased diffusing capacity and small airway dysfunction. While these changes are well recognised in the acute care setting of mechanically ventilated patients receiving FiO₂>50%, little is known about the long-term effect of low flow (24-28%) oxygen. It is widely accepted that the increased survival and quality-of-life benefits of long-term oxygen therapy outweigh the possible risks.

Indeed, there are certain situations in which oxygen therapy is known to have a negative impact on a patient's condition. For example, in a patient who is suffering from paraquat poisoning, oxygen can increase the toxicity. Moreover, oxygen therapy is typically not recommended for patients who have suffered pulmonary fibrosis or other lung damage resulting from bleomycin treatment.

In addition, high levels of oxygen given to infants typically causes blindness by promoting overgrowth of new blood vessels in the eye obstructing sight. This is termed retinopathy of prematurity (ROP). See, e.g., O. D. Saugstaad, Journal of Perinatology (2006) 26, S46-S50.

Exacerbations of Chronic Obstructive Pulmonary Disease COPD

Patients of chronic obstructive pulmonary disease (COPD) often have chronic hypoxaemia with or without CO₂ retention. Oxygen in this situation is required until the exacerbation is settled. While a high FiO₂ of up to 100% can be initially administered in case hypoxemia is severe, it is soon tapered to around 50-60% FiO₂.

As previously discussed, the goal of supplemental oxygen is to maintain a PaO₂ of 55-60 mmHg, which corresponds to SpO₂ of about 90%, since higher concentrations of oxygen can blunt the hypoxic ventilatory drive, which may precipitate hypoventilation and CO₂ retention. Thus, it is advisable to use a regulated flow device such as a venti mask, which guarantees oxygen delivery to a reasonable extent. Once the patient is stabilized, one can shift to nasal prongs—a device that is more comfortable and acceptable to the patient.

Acute Severe Bronchial Asthma

Patients with acute severe asthma or status asthamticus have severe airway obstruction and inflammation. They are generally hypoxemic. Arterial blood sample is immediately obtained and oxygen is started via nasal cannula or preferably via a face mask at flow rate of 4-6 L/min to achieve FiO₂ of 35 to 40%. Higher flow is unlikely to improve oxygenation. Flow rate is adjusted to maintain a PaO₂ of about 80 mmHg or near normal value. Concurrent bronchial hygiene and administration of intravenous fluids, bronchodilators and corticosteroids should alleviate the problems in most of the situations. Administration of sedatives and tranquilizers must be avoided. Sedatives may precipitate CO₂ retention not only in patients with COPD, but also asthma. Assisted ventilation is required in case there is persistence of hypoxemia and/or precipitation of hypercapnia.

Hyperoxia

Oxidative cell injury involves the modification of cellular macromolecules by reactive oxygen intermediates (ROI), often leading to cell death.

Hyperoxia injures cells by virtue of the accumulation of toxic levels of ROI, including H₂O₂ and the superoxide anion (O₂—), which are not adequately scavenged by endogenous antioxidant defences. These oxidants are cytotoxic and have been shown to kill cells via apoptosis, or programmed cell death. If hyperoxia-induced cell death is a result of increased ROI, then O₂ toxicity should kill cells via apoptosis. It has been discovered that hyperoxia kills cells via necrosis, not apoptosis. In contrast, lethal concentrations of either H₂O₂ or O₂-cause apoptosis. Paradoxically, apoptosis is a prominent event in the lungs of animals injured by breathing 100% O₂. These data indicate that O₂ toxicity is somewhat distinct from other forms of oxidative injury and suggest that apoptosis in vivo is not a direct effect of O₂.

Exposure to high oxygen concentration causes direct oxidative cell damage through increased production of reactive oxygen species. In vivo oxygen-induced lung injury is well characterized in rodents and has been used as a valuable model of human respiratory distress syndrome. Hyperoxia-induced lung injury can be considered as a bimodal process resulting (1) from direct oxygen toxicity and (2) from the accumulation of inflammatory mediators within the lungs. Both apoptosis and necrosis have been described in alveolar cells (mainly epithelial and endothelial) during hyperoxia. While the in vitro response to oxygen seems to be cell type-dependent in tissue cultures, it is still unclear which are the death mechanisms and pathways implicated in vivo. Even though it is not yet possible to distinguish unequivocally between apoptosis, necrosis, or other intermediate form(s) of cell death, a great variety of strategies has been shown to prevent alveolar damage and to increase animal survival during hyperoxia.

Oxygen administration can cause structural damage to the lungs. Both proliferative and fibrotic changes of oxygen toxicity have been shown at autopsy on COPD patients treated with long term oxygen. But there is no significant effect of these changes on clinical course or survival of these patients. Most of the structural damage attributable to hyperoxia results from high FiO₂ administration in acute conditions.

With prolonged oxygen therapy there is increase in blood oxygen level, which suppresses peripheral chemoreceptors; depresses ventilator drive and increase in PCO₂. high blood oxygen level may also disrupt the ventilation: perfusion balance (V/Q) and cause an increase in dead space to tidal volume ratio and increase in PCO₂. Therefore, oxygen therapy may accentuate hypoventilation in patients with COPD. This may include hypercapnia and carbon dioxide narcosis. Prehospital hyperoxia from excessive oxygen administration in COPD patients is shown to be dangerous.

An FiO2>0.50 presents a significant risk of absorption atelactasis. N₂ is most plentiful gas in both the alveoli and blood. Breathing high level of O₂ depletes body N₂ levels. As blood N₂ level decreases, total pressure of venous gases rapidly decreases.

Under these conditions, gases within any body cavity rapidly diffuse into venous blood leading to absorption atelactasis. Risk of absorption atelactasis is greatest in patients breathing at low tidal volumes as a result of sedation, surgical pain or central nervous system (CNS) dysfunction. See, e.g., Singh, et al., Supplemental oxygen therapy: Important considerations in oral and maxillofacial surgery, Natl. J. Maxillofac. Surg., 2(1):10-14, Jan.-Jun. 2011.

Role of NO

Nitric oxide is an important signalling molecule in pulmonary vessels. Nitric oxide can moderate pulmonary hypertension caused by elevation of the pulmonary arterial pressure. Inhaling low concentrations of nitric oxide, for example, in the range of 0.01-100 ppm can rapidly and safely decrease pulmonary hypertension in a mammal by vasodilation of pulmonary vessels.

NO has been implicated as both a prooxidant and an antioxidant. One might anticipate, therefore, that the addition of NO in the presence of high inspired O2 might modify the overall response to the high O2 exposure. For example, high O2 increases superoxide production, and superoxide and NO react spontaneously to form peroxynitrite, which can be toxic. Furthermore, oxygen and NO readily combine to form NO₂, which can also be toxic. On the other hand, NO can react with lipid peroxyl radicals to prevent lipid peroxidation, and this might help thwart the increase in lipid peroxidation associated with oxygen toxicity. Furthermore, NO can inhibit neutrophil accumulation and activation. It has been shown that, when endogenous NO production was blocked in neonatal rats, which are relatively O₂-tolerant with Nw-nitro-l-arginine methyl ester, significantly fewer survived exposure to>95%, O₂ compared with control rats, suggesting that endogenous NO has some protective effect.

Inhaled NO was shown to increase survival in high O₂ exposure in rats. The impact of adding NO to high inspired O₂ is clinically relevant because many patients with various forms of acute lung injury, such as adult respiratory distress syndrome, persistent pulmonary hypertension of the newborn caused by meconium aspiration, and so forth, are being treated with inhaled NO while receiving very high fractions of inspired O₂.

In short, using NO allows one to use a reduced amount of supplemental oxygen, thereby reducing oxidative stress, while providing the necessary oxygen enhancement.

A method of providing a therapeutic composition can include administering exogenous NO to modulate the hormesis characteristics of NO. Hormesis in this instance refers to the temporal and dose dependency related to the stimulatory versus inhibitory response to NO. For example, NO stimulates HIF for 30 minutes at low dose during hypoxia. It becomes inhibitory at high doses and after 30 minutes. This suggests that it would be effective to lower doses 0.1 to 5 ppm for up to 30 minutes repeated at a intervals rather than high dose continuous delivery, for example.

Potential Toxicity of NO

Studies have shown that short-term exposure to inhaled NO, O₂ or O₂+ NO increases lung collagen accumulation in neonatal piglets. This may be because NO, unlike O₂ or O₂+ NO, does not induce a concurrent increase in pulmonary matrix degradation. Indeed the increase in lung collagen content found with NO exposure appeared potentially reversible as demonstrated by a significant decline after a 3-day recovery period in RA. The increase in lung collagen accumulation observed with NO represents a finding that NO may have the potential to induce pulmonary fibrosis. Ekekezie, High-dose Inhaled Nitric Oxide and Hyperoxia Increases Lung Collagen Accumulation in Piglets, Biology of the Neonate, 78(3) (2000).

Hydrogen Supplement

Hydrogen gas can act as an antioxidant and is a free radical scavenger. Hydrogen is the most abundant chemical element in the universe, but is seldom regarded as a therapeutic agent. Recent evidence has shown that hydrogen is a potent antioxidative, antiapoptotic and anti-inflammatory agent and so may have potential medical applications in cells, tissues and organs.

Using a mixture of NO and hydrogen gases for inhalation can be useful, for example, during planned coronary interventions or for the treatment of ischemia-reperfusion (I/R) injury. In short, inhaled NO suppresses the inflammation in I/R tissues and hydrogen gas eliminates the adverse by-products of NO exposure, peroxynitrite.

However until applicants' discovery, there has not been a successful combination of hydrogen gas with breathing gas using the claimed apparatus and methods. Applicants have discovered that NO's effect as an antioxidant may be enhanced by eliminating highly reactive by-products of NO inhalation such as peroxynitrite, by adding H2 to inhaled NO gas. Specifically, Applicants found that 1) mice with intratracheal administration of LPS exhibited significant lung injury, which was significantly improved by 2% H₂ and/or 20 ppm NO treatment for 3 hours starting at 5 minutes or 3 hours after LPS administration; 2) H₂ and/or NO treatment inhibited LPS-induced pulmonary early and late NF-κB activation; 3) H₂ and/or NO treatment down-regulated the pulmonary inflammation and cell apoptosis; 4) H₂ and/or NO treatment also significantly attenuated the lung injury in polymicrobial sepsis; and 5) Combination therapy with subthreshold concentrations of H₂ and NO could synergistically attenuate LPS- and polymicrobial sepsis-induced lung injury. In conclusion, these results demonstrate that combination therapy with H₂ and NO could more significantly ameliorate LPS- and polymicrobial sepsis-induced ALI, perhaps by reducing lung inflammation and apoptosis, which may be associated with the decreased NF-κB activity.

Studies have shown that hydrogen gas exhibits cytoprotective effects and transcriptional alterations, and can selectively reduce the generation of hydroxyl radicals and peroxynitrite, thereby protecting the cells against oxidant injury. Yokota, Molecular hydrogen protects chrondrocytes from oxidative stress and indirectly alters gene expressions through reducing peroxynitrite derived from nitric oxide, Medical Gas Research 2015.

In an acute rat model in which oxidative stress was induced in the brain by focal FiO ischemia-reperfusion (I/R), inhaled hydrogen gas markedly suppressed the associated brain injury. Thus it was suggested that administration of hydrogen gas by inhalation may serve as an effective therapy for ischemia-reperfusion, and based on the ability of hydrogen gas to rapidly diffuse across membranes, it can even protect ischemic tissues against oxidative damage. Ohsawa I, et al., Hydrogen acts as a therapeutic antioxidant by selectively reducing cytotoxic oxygen radicals. Nat Med 13: 688-694, 2007.

Breathing NO plus hydrogen gas was also found to reduce cardiac injury and augment recovery of the left ventricular function, by elimination of the nitrotyrosine produced by NO inhalation alone. See, e.g., Shinbo, et al., “Breathing nitric oxide plus hydrogen has reduced ischemia-reperfusion injury and nitrotyrosine production in murine heart,”Am J. Physiol Heart Circ Physiol., 305: H542-H550, 2013. In addition, data has indicated that combination therapy with hydrogen gas and NO can effectively attenuate LPS-induced lung inflammation and injury in mice. Liu, et al, “Combination therapy with NO and H₂ in ALI.”

There are several methods to administer hydrogen, such as inhalation of hydrogen gas, aerosol inhalation of a hydrogen-rich solution, drinking hydrogen dissolved in water, injecting hydrogen-rich saline (HRS) and taking a hydrogen bath. Drinking hydrogen solution (saline/pure water/other solutions saturated with hydrogen) may be more practical in daily life and more suitable for daily consumption. Shen, et al., “A review of experimental studies of hydrogen as a new therapeutic agent in emergency and critical care medicine”Medical Gas Research, 2014. Molecular hydrogen diffuses rapidly across cell membranes, reduces reactive oxygen species, including hydroxyl radicals and peroxynitrite, and suppresses oxidative stress-induced injury in several organs with no known toxicity. Fu, et al., Molecular hydrogen is protective against 6-hydroxydopamine-induced nigrostriatal degeneration in a rat model of Parkinson's disease. Neurosci. Lett. 2009.

Administering Supplemental Oxygen

Supplemental oxygen and NO can be administered by titration. Titration is a method or process of determining the concentration of a dissolved substance in terms of the smallest amount of reagent of known concentration required to bring about a given effect in reaction with a known volume of the test solution.

Any suitable system can be used to deliver NO. NO can be administered by titration. Titration is a method or process of administering a dose of compound such as NO until a visible or detectable change is achieved.

In one embodiment, a nitric oxide delivery system can include a receptacle. A receptacle can include an inlet and an outlet. A receptacle can convert a nitric oxide-releasing agent to nitric oxide (NO). A nitric oxide-releasing agent can include one or more of nitrogen dioxide (NO₂), dinitrogen tetroxide (N₂O₄) or nitrite ions (NO₂⁻). Nitrite ions can be introduced in the form of a nitrite salt, such as sodium nitrite.

A receptacle can include a reducing agent or a combination of reducing agents. A number of reducing agents can be used depending on the activities and properties as determined by a person of skill in the art. In some embodiments, a reducing agent can include a hydroquinone, glutathione, and/or one or more reduced metal salts such as Fe(II), Mo(VI), NaI, Ti(III) or Cr(III), thiols, or NO₂⁻. A reducing agent can include 3,4 dihydroxy-cyclobutene-dione, maleic acid, croconic acid, dihydroxy-fumaric acid, tetra-hydroxy-quinone, p-toluene-sulfonic acid, tricholor-acetic acid, mandelic acid, 2-fluoro-mandelic acid, or 2,3,5,6-tetrafluoro-mandelic acid. A reducing agent can be safe (i.e., non-toxic and/or non-caustic) for inhalation by a mammal, for example, a human. A reducing agent can be an antioxidant. An antioxidant can include any number of common antioxidants, including ascorbic acid, alpha tocopherol, and/or gamma tocopherol. A reducing agent can include a salt, ester, anhydride, crystalline form, or amorphous form of any of the reducing agents listed above. A reducing agent can be used dry or wet. For example, a reducing agent can be in solution. A reducing agent can be at different concentrations in a solution. Solutions of the reducing agent can be saturated or unsaturated. While a reducing agent in organic solutions can be used, a reducing agent in an aqueous solution is preferred. A solution including a reducing agent and an alcohol (e.g. methanol, ethanol, propanol, isopropanol, etc.) can also be used.

A receptacle can include a support. A support can be any material that has at least one solid or non-fluid surface (e.g. a gel). It can be advantageous to have a support that has at least one surface with a large surface area. In preferred embodiments, the support can be porous or permeable. One example of a support can be surface-active material, for example, a material with a large surface area that is capable of retaining water or absorbing moisture. Specific examples of surface active materials can include silica gel or cotton. The term “surface-active material” denotes that the material supports an active agent on its surface.

A support can include a reducing agent. Said another way, a reducing agent can be part of a support. For example, a reducing agent can be present on a surface of a support. One way this can be achieved can be to coat a support, at least in part, with a reducing agent. In some cases, a system can be coated with a solution including a reducing agent. Preferably, a system can employ a surface-active material coated with an aqueous solution of antioxidant as a simple and effective mechanism for making the conversion. Generation of NO from a nitric oxide-releasing agent performed using a support with a reducing agent can be the most effective method, but a reducing agent alone can also be used to convert nitric oxide-releasing agent to NO.

In some circumstances, a support can be a matrix or a polymer, more specifically, a hydrophilic polymer. A support can be mixed with a solution of the reducing agent. The solution of reducing agent can be stirred and strained with the support and then drained. The moist support-reducing agent mixture can be dried to obtain the proper level of moisture. Following drying, the support-reducing agent mixture may still be moist or may be dried completely. Drying can occur using a heating device, for example, an oven or autoclave, or can occur by air drying.

In general, a nitric oxide-releasing agent can be converted to NO by bringing a gas including the nitric oxide-releasing agent in contact with a reducing agent. In one example, a gas including a nitric oxide-releasing agent can be passed over or through a support including a reducing agent. When the reducing agent is ascorbic acid (i.e. vitamin C), the conversion of nitrogen dioxide to nitric oxide can be quantitative at ambient temperatures.

The generated nitric oxide can be delivered to a mammal, which can be a human. To facilitate delivery of the nitric oxide, a system can include a patient interface. Examples of a patient interface can include a mouth piece, nasal cannula, face mask, fully-sealed face mask or an endotracheal tube. A patient interface can be coupled to a delivery conduit. A delivery conduit can include a ventilator or an anesthesia machine.

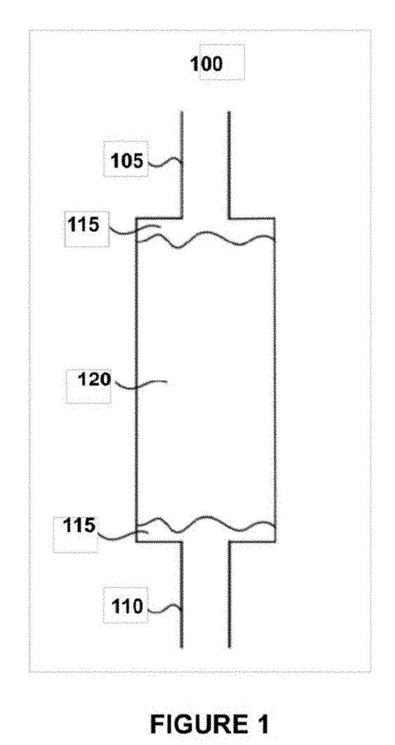

FIG. 1 illustrates one embodiment of a receptacle for generating NO by converting a nitric oxide-releasing agent to NO. The receptacle 100 can include an inlet 105 and an outlet 110. An example of a receptacle can be a cartridge. A cartridge can be inserted into and removed from an apparatus, platform or system. Preferably, a cartridge is replaceable in the apparatus, platform or system, and more preferably, a cartridge can be disposable. Screen and glass wool 115 can be located at either or both of the inlet 105 and the outlet 110. The remainder of the receptacle 100 can include a support. In a preferred embodiment, a receptacle 100 can be filled with a surface-active material 120. The surface-active material 120 can be soaked with a saturated solution of antioxidant in water to coat the surface-active material. The screen and glass wool 115 can also be soaked with the saturated solution of antioxidant in water before being inserted into the receptacle 100. In general, a process for converting a nitric oxide-releasing agent to NO can include passing a gas including a nitric oxide-releasing agent into the inlet 105. The gas can be communicated to the outlet 110 and into contact with a reducing agent. In a preferred embodiment, the gas can be fluidly communicated to the outlet 110 through the surface-active material 120 coated with a reducing agent. As long as the surface-active material remains moist and the reducing agent has not been used up in the conversion, the general process can be effective at converting a nitric oxide-releasing agent to NO at ambient temperature.

The inlet 105 may receive the gas including a nitric oxide-releasing agent from a gas pump that fluidly communicates the gas over a diffusion tube or a permeation cell. The inlet 105 also may receive the gas including a nitric oxide-releasing agent, for example, from a pressurized bottle of a nitric oxide-releasing agent. A pressurized bottle may also be referred to as a tank. The inlet 105 also may receive a gas including a nitric oxide-releasing agent can be NO₂ gas in nitrogen (N₂), air, or oxygen (O₂). A wide variety of flow rates and NO₂ concentrations have been successfully tested, ranging from only a few ml per minute to flow rates of up to 5,000 ml per minute.

The conversion of a nitric oxide-releasing agent to NO can occur over a wide range of concentrations of a nitric oxide-releasing agent. For example, experiments have been carried out at concentrations in air of from about 2 ppm NO₂ to 100 ppm NO₂, and even to over 1000 ppm NO₂. In one example, a receptacle that was approximately 6 inches long 16250.0040 and had a diameter of1.5-inches was packed with silica gel that had first been soaked in a saturated aqueous solution of ascorbic acid. The moist silica gel was prepared using ascorbic acid designated as A.C.S reagent grade 99.1% pure from Aldrich Chemical Company and silica gel from Fischer Scientific International, Inc., designated as S8 32-1, 40 of Grade of 35 to 70 sized mesh. Other sizes of silica gel can also be effective. For example, silica gel having an eighth-inch diameter can also work.

In another example, silica gel was moistened with a saturated solution of ascorbic acid that had been prepared by mixing 35% by weight ascorbic acid in water, stirring, and straining the water/ascorbic acid mixture through the silica gel, followed by draining. The conversion of NO₂ to NO can proceed well when the support including the reducing agent, for example, silica gel coated with ascorbic acid, is moist. In a specific example, a receptacle filled with the wet silica gel/ascorbic acid was able to convert 1000 ppm of NO₂ in air to NO at a flow rate of 150 ml per minute, quantitatively, non-stop for over 12 days.

A receptacle can be used for inhalation therapy. In addition to converting a nitric oxide-releasing agent to nitric oxide to be delivered during inhalation therapy, a receptacle can remove any NO₂ that chemically forms during inhalation therapy (e.g., nitric oxide that is oxidized to form nitrogen dioxide). In one such example, a receptacle can be used as a NO₂ scrubber for NO inhalation therapy that delivers NO from a pressurized bottle source. A receptacle may be used to help ensure that no harmful levels of NO₂ are inadvertently inhaled by the patient.

In addition, a receptacle may be used to supplement or replace some or all of the safety devices used during inhalation therapy in conventional NO inhalation therapy. For example, one type of safety device can warn of the presence of NO₂ in a gas when the concentration of NO₂ exceeds a preset or predetermined limit, usually 1 part per million or greater of NO₂. Such a safety device may be unnecessary when a receptacle is positioned in a NO delivery system just prior to the patient breathing the NO laden gas. A receptacle can convert any NO₂ to NO just prior to the patient breathing the NO laden gas, making a device to warn of the presence of NO₂ in gas unnecessary.

Furthermore, a receptacle placed near the exit of inhalation equipment, gas lines or gas tubing can also reduce or eliminate problems associated with formation of NO₂ that occur due to transit times in the equipment, lines or tubing. As such, use of a receptacle can reduce or eliminate the need to ensure the rapid transit of the gas through the gas plumbing lines that is needed in conventional applications. Also, a receptacle can allow the NO gas to be used with gas balloons to control the total gas flow to the patient.

Alternatively or additionally, a NO₂ removal receptacle can be inserted just before the attachment of the delivery system to the patient to further enhance safety and help ensure that all traces of the toxic NO₂ have been removed. The NO₂ removal receptacle may be a receptacle used to remove any trace amounts of NO₂. Alternatively, the NO₂ removal receptacle can include heat-activated alumina. A receptacle with heat-activated alumina, such as supplied by Fisher Scientific International, Inc., designated as ASOS-212, of 8-14 sized mesh can be effective at removing low levels of NO₂ from an air or oxygen stream, and yet, can allow NO gas to pass through without loss. Activated alumina, and other high surface area materials like it, can be used to scrub NO₂ from a NO inhalation line.

In another example, a receptacle can be used to generate NO for therapeutic gas delivery. Because of the effectiveness of a receptacle in converting nitric oxide-releasing agents to NO, nitrogen dioxide (gaseous or liquid) or dinitrogen tetroxide can be used as the source of the NO. When nitrogen dioxide or dinitrogen tetroxide is used as a source for generation of NO, there may be no need for a pressurized gas bottle to provide NO gas to the delivery system. By eliminating the need for a pressurized gas bottle to provide NO, the delivery system may be simplified as compared with a conventional apparatus that is used to deliver NO gas to a patient from a pressurized gas bottle of NO gas. A NO delivery system that does not use pressurized gas bottles may be more portable than conventional systems that rely on pressurized gas bottles.

In some delivery systems, the amount of nitric oxide-releasing agent in a gas can be approximately equivalent to the amount of nitric oxide to be delivered to a patient. For example, if a therapeutic dose of 20 ppm of nitric oxide is to be delivered to a patient, a gas including 20 ppm of a nitric oxide-releasing agent (e.g., NO₂) can be released from a gas bottle or a diffusion tube. The gas including 20 ppm of a nitric oxide-releasing agent can be passed through one or more receptacles to convert the 20 ppm of nitric oxide-releasing agent to 20 ppm of nitric oxide for delivery to the patient. However, in other delivery systems, the amount of nitric oxide-releasing agent in a gas can be greater than the amount of nitric oxide to be delivered to a patient. For example, a gas including 800 ppm of a nitric oxide-releasing agent can be released from a gas bottle or a diffusion tube. The gas including 800 ppm of a nitric oxide-releasing agent can be passed through one or more receptacles to convert the 800 ppm of nitric oxide-releasing agent to 800 ppm of nitric oxide. The gas including 800 ppm of nitric oxide can then be diluted in a gas including oxygen (e.g., air) to obtain a gas mixture with 20 ppm of nitric oxide for delivery to a patient. Traditionally, the mixing of a gas including nitric oxide with a gas including oxygen to dilute the concentration of nitric oxide has occurred in a line or tube of the delivery system. The mixing of a gas including nitric oxide with a gas including oxygen can cause problems because nitrogen dioxide can form. To avoid this problem, two approaches have been used. First, the mixing of the gases can be performed in a line or tube immediately prior to the patient interface, to minimize the time nitric oxide is exposed to oxygen, and consequently, reduce the nitrogen dioxide formation. Second, a receptacle can be placed at a position downstream of the point in the line or tubing where the mixing of the gases occurs, in order to convert any nitrogen dioxide formed back to nitric oxide.

While these approaches can minimize the nitrogen dioxide levels in a gas delivered to a patient, these approaches have some drawbacks. Significantly, both of these approaches mix a gas including nitric oxide with a gas including oxygen in a line or tubing of the system. One problem can be that lines and tubing in a gas delivery system can have a limited volume, which can constrain the level of mixing. Further, a gas in lines and tubing of a gas delivery system can experience variations in pressure and flow rates. Variations in pressure and flow rates can lead to an unequal distribution of the amount each gas in a mixture throughout a delivery system. Moreover, variations in pressure and flow rates can lead to variations in the amount of time nitric oxide is exposed to oxygen within a gas mixture. One notable example of this arises with the use of a ventilator, which pulses gas through a delivery system. Because of the variations in pressure, variations in flow rates and/or the limited volume of the lines or tubing where the gases are mixed, a mixture of the gases can be inconsistent, leading to variation in the amount of nitric oxide, nitrogen dioxide, nitric oxide-releasing agent and/or oxygen between any two points in a delivery system.

To address these problems, a mixing chamber can also be used to mix a first gas and a second gas. A first gas can include oxygen; more specifically, a first gas can be air. A second gas can include a nitric oxide-releasing agent and/or nitric oxide. A first gas and a second gas can be mixed within a chamber to form a gas mixture. The mixing can be an active mixing performed by a mixer within a chamber. For example, a mixer can be a moving support. The mixing within a receptacle can also be a passive mixing, for example, the result of diffusion.

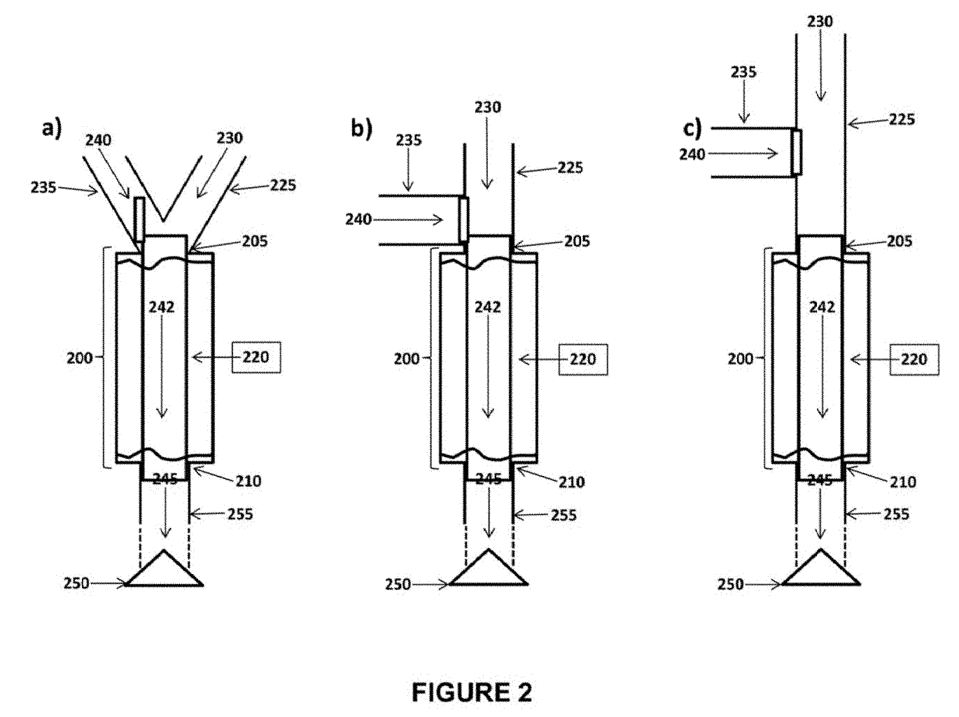

As shown in FIGS. 2a, 2b and 2c, a receptacle 200 can be coupled to a gas conduit 225. A first gas 230 including oxygen can be communicated through a gas conduit 225 to the receptacle 200. The communication of the first gas through the gas conduit can be continuous or it can be intermittent. For instance, communicating the first gas intermittently can include communicating the first gas through the gas conduit in one or more pulses. Intermittent communication of the first gas through gas conduit can be performed using a gas bag, a pump, a hand pump, an anesthesia machine or a ventilator.

A gas conduit can include a gas source. A gas source can include a gas bottle, a gas tank, a permeation cell or a diffusion tube. Nitric oxide delivery systems including a gas bottle, a gas tank a permeation cell or a diffusion tube are described, for example, in U.S. Pat. Nos. 7,560,076 and 7,618,594, each of which are incorporated by reference in its entirety. Alternatively, a gas source can include a reservoir and restrictor, as described in U.S. patent application Ser. Nos. 12/951,811, 13/017,768 and 13/094,535, each of which is incorporated by reference in its entirety. A gas source can include a pressure vessel, as described in U.S. patent application Ser. No. 13/492,154, which is incorporated by reference in its entirety. A gas conduit can also include one or more additional receptacles. Additional components including one or more sensors for detecting nitric oxide levels, one or more sensors for detecting nitrogen dioxide levels, one or more sensor for detecting oxygen levels, one or more humidifiers, valves, tubing or lines, a pressure regulator, flow regulator, a calibration system and/or filters can also be included in a gas conduit.

A second gas 240 can also be communicated to a chamber 200. A second gas can be supplied into a gas conduit, as shown in FIGS. 2b and 2c. Preferably, a second gas 240 can be supplied into a gas conduit 225 immediately prior to a chamber 200, as shown in FIG. 2b. A second gas 240 can be supplied into a gas conduit 225 via a second gas conduit 235, which can join or be coupled to the gas conduit 225. Once a second gas 240 is supplied into a gas conduit 225, both the first gas 230 and the second gas 240 can be communicated in the inlet 205 of a chamber 200 for mixing. Alternatively, a second gas 240 can be supplied at a receptacle 200, as show in FIG. 2a. For example, a second gas 240 can be supplied directly into the inlet 205 of a chamber 200.

Once a first gas 230 and a second gas 240 are within a chamber 200, a first gas 230 and a second gas 240 can mix to form a gas mixture 242 including oxygen and one or more of nitric oxide, a nitric oxide-releasing agent (which can be nitrogen dioxide) and nitrogen dioxide. The gas mixture 242 can contact a reducing agent, which can be on a support 220 within the receptacle. The reducing agent can convert nitric oxide-releasing agent and/or nitrogen dioxide in the gas mixture to nitric oxide.

The gas mixture including nitric oxide 245 can then be delivered to a mammal, most preferably, a human patient. The concentration of nitric oxide in a gas mixture can be at least 0.01 ppm, at least 0.05 ppm, at least 0.1 ppm, at least 0.5 ppm, at least 1 ppm, at least 1.5 ppm, at least 2 ppm or at least 5 ppm. The concentration of nitric oxide in a gas mixture can be at most 100 ppm, at most 80 ppm, at most 60 ppm, at most 40 ppm, at most 25 ppm, at most 20 ppm, at most 10 ppm, at most 5 ppm or at most 2 ppm.

Delivering the gas mixture including nitric oxide from the receptacle 200 to the mammal can include passing the gas mixture through a delivery conduit. A delivery conduit 255 can be located between the receptacle 200 and a patient interface 250. In some embodiments, a delivery conduit 255 can be coupled to the outlet 210 of a receptacle 200 and/or coupled to the patient interface 250. As indicated by the dashed lines in FIGS. 2a, 2b and 2c, a delivery conduit can include additional components, for example, a humidifier or one or more additional receptacles.

Delivery of a gas mixture can include continuously providing the gas mixture to the mammal. When the delivery of the gas mixture includes continuously providing the gas mixture to the mammal, the volume of the receptacle can be greater than the volume of the delivery conduit. The larger volume of the receptacle can help to ensure that the gas mixture is being thoroughly mixed prior to delivery. Generally, more complete mixing can occur as the ratio of the volume of the receptacle to the volume of the delivery conduit increases. A preferable level of mixing can occur when the volume of the receptacle is at least twice the volume of the delivery conduit. The volume of the receptacle can also be at least 1.5 times, at least 3 times, at least 4 times or at least 5 times the volume of the delivery conduit.

When the volume of the receptacle is greater than the volume of the delivery conduit or the volume of gas mixture in the delivery conduit, the gas mixture may not go directly from the receptacle to the mammal, but instead, can be delayed in the receptacle or delivery conduit. It is this delay that can provide the time needed to mix the gas so that the NO concentration remains constant within a breath.

This delay can result in the storage of the gas mixture in the receptacle. The gas mixture can be stored in the receptacle for a predetermined period of time. The predetermined period of time can be at least 1 second, at least 2 seconds, at least 6 seconds, at least 10 seconds, at least 20 seconds, at least 30 seconds or at least 1 minute.

The mixing that occurs due to the delay of the gas mixture (i.e. storage of the gas mixture in a receptacle) can be so effective that the intra-breath variation can be identical to what could be achieved under ideal conditions when premixed gas was provided. This can be referred to as “perfect mixing.” For continuous delivery, this can mean that the concentration of nitric oxide in the gas mixture delivered to a mammal remains constant over a period of time (e.g. at least 1 min, at least 2 min, at least 5 min, at least 10 min or at least 30 min). For a concentration to remain constant, the concentration can remain with a range of at most±10%, at most±5%, or at most±2% of a desired concentration for delivery.

Delivery of the gas mixture can include intermittently providing the gas mixture to the mammal. Intermittent delivery of a gas mixture can be the result of intermittent communication of a first or second gas into the system. Said another way, intermittent communication of a first or second gas through a gas conduit can result in an increased area of pressure, which can traverse into the receptacle causing intermittent communication of the gas mixture. Intermittent delivery can be performed using a gas bag, a pump, a hand pump, an anesthesia machine or a ventilator.

The intermittent delivery can include an on-period, when the gas mixture is delivered to a patient, and an off-period, when the gas mixture is not delivered to a patient. Intermittent delivery can include delivering one or more pules of the gas mixture.

An on-period or a pulse can last for a few seconds up to as long as several minutes. In one embodiment, an on-period or a pulse can last for 1, 5, 10, 15, 20, 25, 30, 35, 40, 45, 50, 55 or 60 seconds. In another embodiment, the on-period or a pulse can last for 1, 2, 3, 4 or 5 minutes. In a preferred embodiment, an on-period or a pulse can last for 0.5-10 seconds, most preferably 1-6 seconds.

Intermittent delivery can include a plurality of on-periods or pulses. For example, intermittent delivery can include at least 1, at least 2, at least 5, at least 10, at least 50, at least 100 or at least 1000 on-periods or pulses.

The timing and duration of each on-period or pulse of the gas mixture can be pre-determined. Said another way, the gas mixture can be delivered to a patient in a pre-determined delivery sequence of one or more on-periods or pulses. This can be achieved using an anesthesia machine or a ventilator, for example.

When the delivery of the gas mixture includes intermittently providing the gas mixture to the mammal, the volume of the receptacle can be greater than the volume of the gas mixture in a pulse or on-period. The larger volume of the receptacle can help to ensure that the gas mixture is being thoroughly mixed prior to delivery. Generally, more complete mixing can occur as the ratio of the volume of the receptacle to the volume of the gas mixture in a pulse or on-period delivered to a mammal increases. A preferable level of mixing can occur when the volume of the receptacle is at least twice the volume of the gas mixture in a pulse or on-period. The volume of the receptacle can also be at least 1.5 times, at least 3 times, at least 4 times or at least 5 times the volume of the gas mixture in a pulse or on-period.

When the volume of the receptacle is greater than the volume of the volume of the gas mixture in a pulse or on-period, the gas mixture may not go directly from the receptacle to the mammal, but instead, can be delayed in the receptacle or delivery conduit for one or more pulses or on-periods. It is this delay that can provide the time needed to mix the gas so that the NO concentration remains constant between delivered pulses or on-periods.

In addition to storage as a result of off-periods, the delay caused by the differing volumes can result in the storage of the gas mixture in the receptacle. The gas mixture can be stored in the receptacle for a predetermined period of time. The predetermined period of time can be during or between pulses or on-periods. The predetermined period of time can be at least 1 second, at least 2 seconds, at least 6 seconds, at least 10 seconds, at least 20 seconds, at least 30 seconds or at least 1 minute.

The mixing that occurs due to the delay of the gas mixture (i.e. storage of the gas mixture in a receptacle) can be so effective that the intra-breath variation can be identical to what could be achieved under ideal conditions when premixed gas was provided. Intermittent delivery an include providing the gas mixture for two or more pulses or on-periods. Using intermittent delivery, the concentration of nitric oxide in each pulse or on-period can vary by less than 10%, by less than 5%, or by less than 2%. In other words, the variation between the concentration of nitric oxide in a first pulse and the concentration of nitric oxide in a second pulse is less than 10% (or less than 5% or 2%) of the concentration of nitric oxide in the first pulse. In another embodiment, using intermittent delivery, the concentration of nitric oxide in each pulse or on-period can vary by less than 10 ppm, less than 5 ppm, less than 2 ppm or less than 1 ppm. Said another way, the difference between the concentration of nitric oxide in a first pulse and the concentration of nitric oxide in a second pulse is less than 10 ppm, less than 5 ppm, less than 2 ppm or less than 1 ppm.

### EXAMPLES

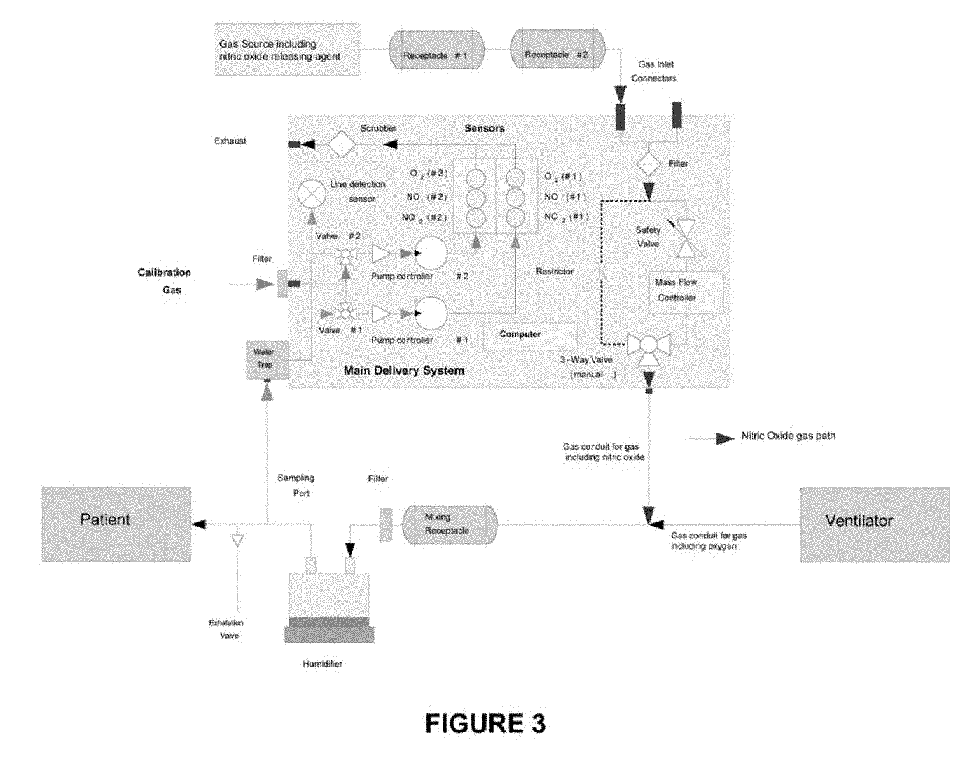

FIG. 3 shows the flow path schematics of an embodiment of a system where a receptacle is used for mixing gas. In this configuration, the gas source including a nitric oxide-releasing agent can be NO₂ in air, for example a bottle of 800 ppm NO₂ in air. Alternatively, the gas source can also be from a liquid source. If a liquid source is used, then the concentration of the source can be variable. In some instances, the concentration of NO₂ can be from about 1000 ppm down to about 50 ppm. The concentration of NO₂ from a liquid source can be controlled by controlling the temperature of the source.

The embodiment shown in FIG. 3 has demonstrated the ability to supply a constant concentration of NO for the duration of the inspired breath. The functions of a receptacle, shown as a mixing receptacle in FIG. 3, can include:

1) To convert any NO₂ that may have formed in the line into NO.

2) To provide adequate mixing of NO in the patient circuit prior to inhalation.

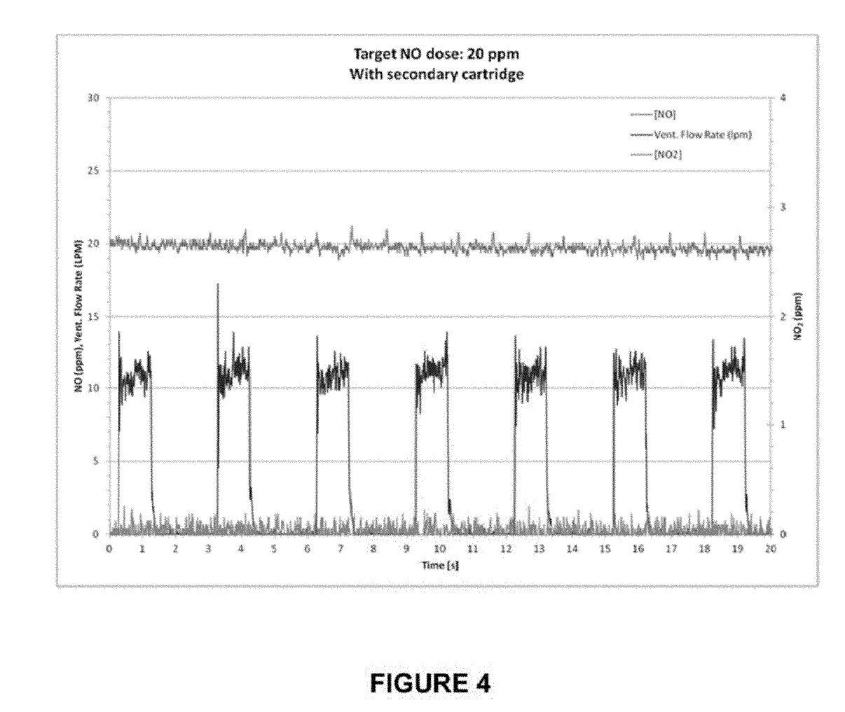

FIG. 4 shows a typical response of a system as embodied in FIG. 3 configured to deliver 20 ppm of NO. The NO₂ values (bottom) are shown (right hand axis). These measurements were obtained using the electrochemical gas analyzers that are part of the system. It is to be noted that the NO₂ levels can be essentially zero when the NO level is at 20 ppm. As shown by the middle plot, the ventilator flow rate is shown (left hand axis). To focus on the worst case scenario, the bias flow of the ventilator was set to zero.

The system was delivering 20 ppm of NO in 21% oxygen using an infant ventilator (Bio-Med Devices CV2+) with the ventilator settings shown in Table 1. The slower breathing rate was used as the worst case for NO mixing, because of the longer pause during exhalation.

TABLE 1
Ventilator Settings
Ventilator Settings

| | Pressure |
|---|---|
| Mode | Control |
| Rate (BPM) | 40 |
| Inspiratory Time INSP (sec) | 0.50 |
| Flow (LPM) | 6.0 |
| I:E Ratio | 1:2.0 |

The NO measurements were within product specifications (±20%). The conversion of NO₂ to NO in the receptacle overcomes the formation of NO₂ that is caused by the delay due to mixing.

As discussed above, the mixing can occur if the volume of the receptacle exceeds the ventilator pulse volume. For example, a 6000 ml/min and 40 breaths per minute the volume of the pulse is 150 ml. Good mixing can occur as long as the volume of the mixing chamber is greater than twice this volume.

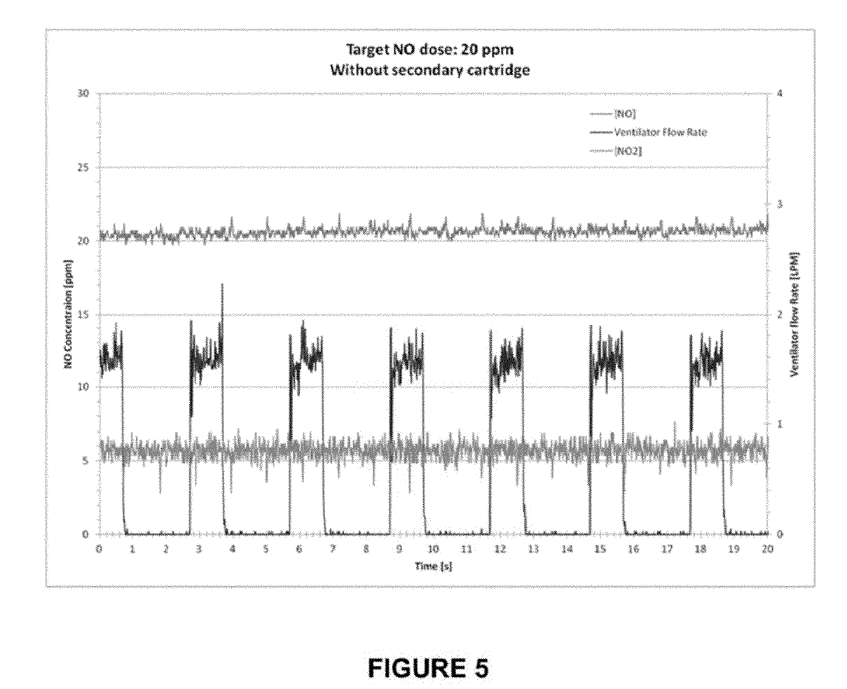

On the other hand, FIG. 5 shows the same response but without the receptacle, shown as the mixing receptacle in FIG. 3, in line with the patient. The NO₂ levels read around 0.6 ppm, which would be unacceptable for a neonate. The receptacle converts all of the NO₂ that was formed back into NO. These two figures clearly demonstrate the effect of a receptacle for converting NO₂ into NO, namely the receptacle reduced the NO₂ level as measured at the patient from 0.6 to 0 ppm.

The mixing performance of the receptacle was assessed using a high speed chemiluminescence detector with a 90% rise time of 250 msec. A very high speed NO detector was needed to catch the intra-breath variability of nitric oxide.

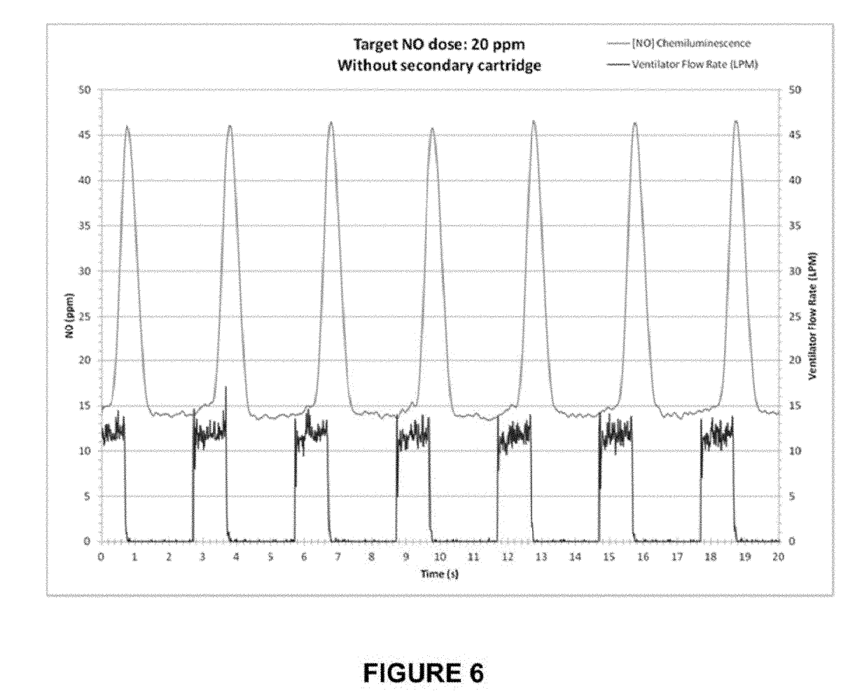

FIG. 6 shows the response of the system without the receptacle for mixing the gases (no mixing function). This chart shows the high speed version of the NO waveform presented in FIG. 5. The bottom line shows the flow rate of the ventilator. As can be seen, the absence of the receptacle introduced spikes of 30 ppm of nitric oxide (top) during the inspiratory time. Intra-breath variability of this magnitude is unacceptable.

Previous technology partially solved this problem by tracking the rapid intra-breath flow changes in the ventilator circuit and uses the electronic signal from the flow sensor to synchronize the valve that introduces the NO into the circuit. This is a difficult and complex electronic solution that requires high speed sensors and very fast computer algorithms operating in real time. Because it is so difficult to execute, the FDA (in their Guidance document) allows the NO to vary from 0 to 150% of the mean, if the total duration of these transient concentrations did not exceed 10% of the volumetric duration of the breath.

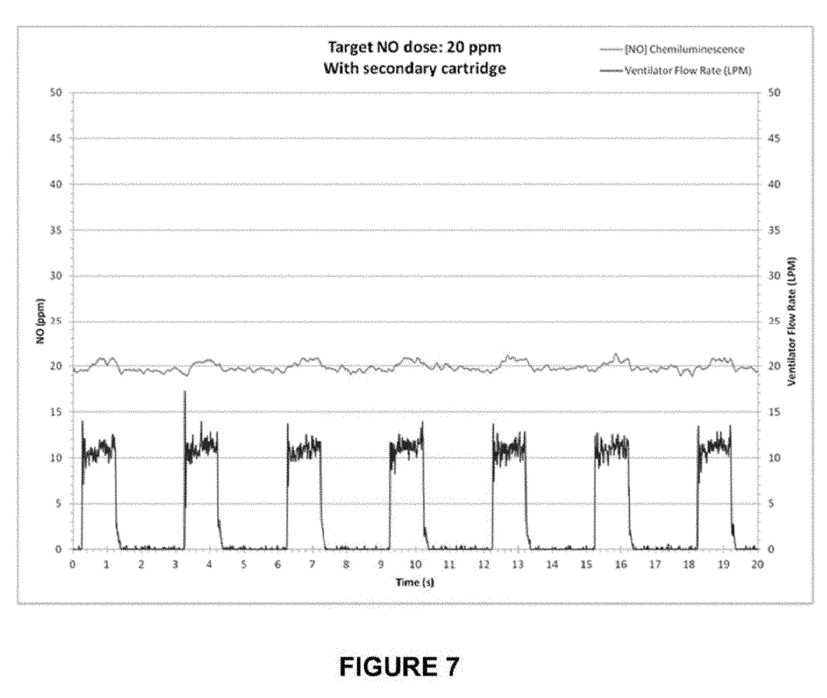

FIG. 7 shows the high speed NO version of FIG. 4 including a receptacle. The high speed detector was able to detect intra-breath variations as low as 1 ppm for the same ventilator settings used in FIG. 6. (In FIG. 4, the pulsations are not shown on the NO reading since the time response of the electro-chemical cell and associated electronics was significantly greater than the time between breaths.) The only difference was the addition of the receptacle which provides the mixing function.

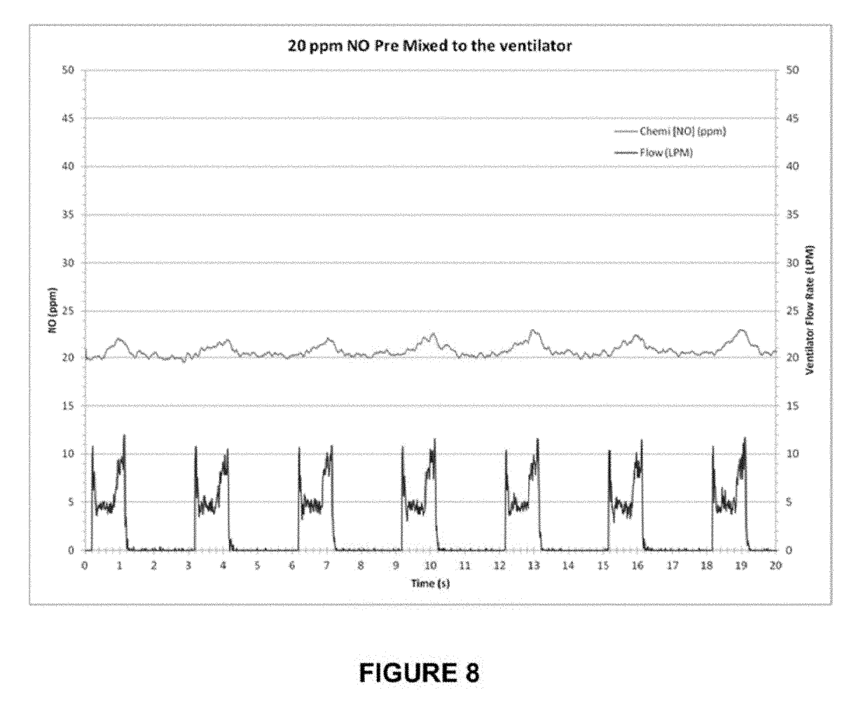

Ideal mixing can happen when the NO gas is premixed and delivered directly using the ventilator. This perfect mixing condition can provide a baseline in order to validate chemiluminescence measurements under pulsing conditions. A blender was used to premix 800 ppm of NO with air to generate a 20 ppm gas to be delivered using a ventilator only. Chemiluminescence was used to measure the NO delivered to the artificial lung. FIG. 8 shows the results. From the peaks in the NO plot (top), it is evident that the chemiluminescence device was affected by the pulsing nature of the flow (bottom). The NO measurements were almost flat but some variations were still present.

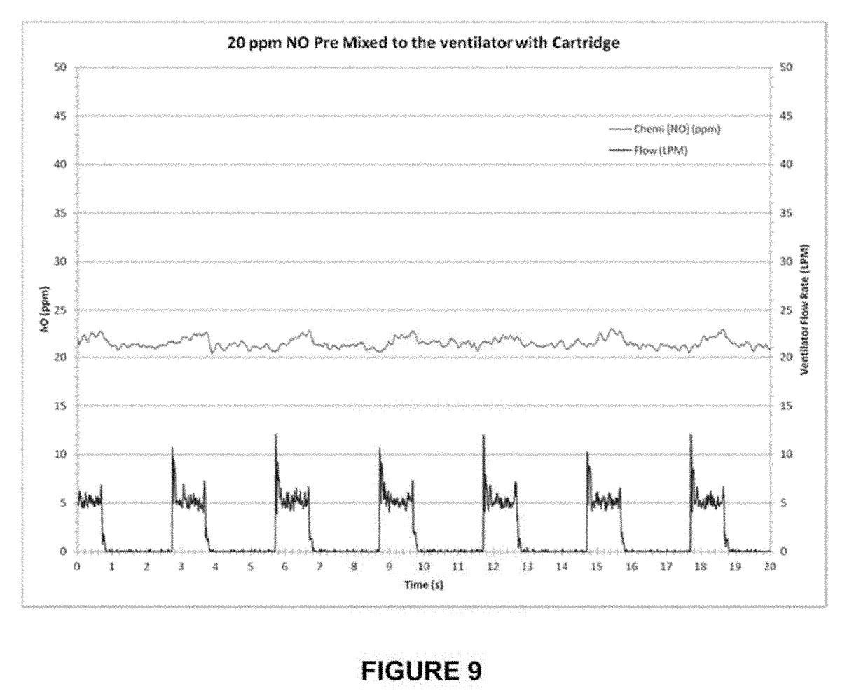

FIG. 9 shows the same experiment but the system includes a receptacle within the breathing circuit. The small amplitude oscillations were present in the NO measurements (top). From these simple experiments, it was concluded that the pulsing flow from the ventilator can provide a perfectly flat NO response using the chemiluminescence device. Furthermore, these oscillations may be due to the pressure changes in the breathing circuit since they were synchronized with the ventilator flow rate measurements (bottom). The intra breath variation that was achieved by mixing in the cartridge was indistinguishable from ideal and what can be achieved using premixed gases. In addition, the NO₂ impurity level is reduced to almost 0.0 ppm.

Constant NO injection into the breathing circuit can be a simple and viable technique as long as a receptacle is both a mixer with sufficient volume and can remove NO₂ from the circuit or can convert the NO₂ back into NO.

FIG. 10 shows an embodiment of the invention as described in more detail above.

Details of one or more embodiments are set forth in the accompanying drawings and description. Other features, objects, and advantages will be apparent from the description, drawings, and claims. Although a number of embodiments of the invention have been described, it will be understood that various modifications may be made without departing from the spirit and scope of the invention. It should also be understood that the appended drawings are not necessarily to scale, presenting a somewhat simplified representation of various features and basic principles of the invention.

What is claimed:

1. A method of providing a therapeutic composition comprising:
administering a ROS reducing drug;
administering exogenous nitric oxide; and
reducing symptoms of oxidative stress and/or fibrosis in a patient.

2. The method of claim 1 further comprising
mixing a first gas including oxygen and a second gas including a nitric oxide-releasing agent within a receptacle to form a gas mixture, wherein the receptacle includes an inlet, an outlet and a reducing agent; and
contacting the nitric oxide-releasing agent in the gas mixture with the reducing agent to generate nitric oxide

3. The method of claim 1 wherein administering exogenous NO is in combination with a prostacyclin drug, anti-fibrotic drug, anti-hypertensive drug, or calcium channel blocker.

4. The method of claim 1 wherein the exogenous NO is inhaled.

5. The method of claim 1 wherein the symptoms of oxidative stress include memory loss and/or brain fog.

6. The method of 1, wherein the symptoms of oxidative stress include fatigue.

7. The method of 1, wherein the symptoms of oxidative stress include muscle and/or joint pain.

8. The method of 1, wherein the symptoms of oxidative stress include decreased eye sight.

9. The method of claim 1 wherein the symptoms of oxidative stress include headaches and sensitivity to noise.

10. The method of claim 1 wherein the symptoms of oxidative stress include susceptibility to infections.

11. The method of claim 1, wherein the nitric oxide-releasing agent is nitrogen dioxide.

12. The method of claim 1, further comprising delivering a hydrogen gas.

13. The method of claim 12, wherein the hydrogen acts to eliminate peroxynitrite, thereby reducing adverse effects of nitric oxide.

14. The method of claim 1, wherein the ROS is a result of a disease.

15. The method of claim 1, wherein the ROS is drug-induced.

16. The method of any one of claim 1, wherein the second gas includes an inert gas or oxygen.

17. The method of claim 1, wherein the concentration of nitric oxide in the gas mixture delivered is at least 0.01 ppm and at most 2 ppm.

18. The method of claim 1, wherein the patient is treated for symptoms of interstitial lung disease, oxygen-induced inflammation, cardiac ischemia, myocardial dysfunction, ARDS, pneumonia, pulmonary embolism, COPD, emphysema, fibrosis, sleep apnea or mountain sickness due to high altitude.

19. The method of claim 2, wherein delivering the gas mixture including nitric oxide from the receptacle to the mammal includes passing the gas mixture through a delivery conduit located between the receptacle and a patient interface.

20. The method of claim 19, wherein the volume of the receptacle is greater than the volume of the delivery conduit.

21. The method of claim 19, wherein the volume of the receptacle is at least two times the volume of the delivery conduit.

22. The method of claim 2, wherein delivering the gas mixture including nitric oxide from the receptacle to the mammal includes intermittently providing the gas mixture to the mammal.

23. The method of claim 2, wherein delivering the gas mixture including nitric oxide from the receptacle to the mammal includes pulsing the gas mixture.

24. The method of claim 23, wherein pulsing includes providing the gas mixture for one or more pulses of 1 to 6 seconds.

25. The method of claim 23, wherein the volume of the receptacle is greater than the volume of the gas mixture in a pulse.

26. The method of claim 23, wherein the volume of the receptacle is at least twice the volume of the gas mixture in a pulse.

27. The method of claim 23, wherein the gas mixture is stored in the receptacle between pulses.

28. The method of claim 2, comprising storing the gas mixture in the receptacle for a predetermined period of time, and wherein the predetermined period is at least 1 second.

29. The method of claim 23, wherein pulsing includes providing the gas mixture for two or more pulses and the concentration of nitric oxide in each pulse varies by less than 10%.

30. The method of claim 23, wherein pulsing includes providing the gas mixture for two or more pulses and the concentration of nitric oxide in each pulse varies by less than 10 ppm.

31. The method of claim 2, comprising communicating the first gas through a gas conduit to the receptacle and supplying the second gas into the gas conduit immediately prior to the receptacle.

32. The method of claim 2, comprising supplying the second gas at the receptacle.

33. The method of claim 1, further comprising administering exogenous NO in an amount effective to modulate the hormesis characteristics of NO.

34. The method of claim 1 wherein the nitric oxide is provided in an effective amount to minimize hemolysis during sepsis.

35. The method of claim 1, wherein the nitric oxide is administered to neonates.

36. The method of claim 1, wherein the nitric oxide is administered to pediatric patients.

37. The method of claim 1, wherein the nitric oxide is administered to adults.

38. The method of claim 1, wherein the nitric oxide is provided through a cartridge having a length, width, and thickness, an outer surface, and an inner surface, and can be substantially cylindrical in shape.

39. The method of claim 38, wherein the thickness between the inner and outer surface is constant, thereby providing a uniform exposure to the reducing agents.

40. The method of claim 38, wherein the cartridge is configured to utilize the whole surface area in converting nitric oxide-releasing agents to NO.

41. A method of providing a therapeutic composition comprising:
administering an anti-fibrotic drug;
administering inhaled nitric oxide; and
reducing symptoms of oxidative stress and/or fibrosis in a patient.

42. A method of providing a therapeutic composition comprising:
administering a calcium channel blocker;
administering inhaled nitric oxide; and
reducing symptoms of oxidative stress and/or fibrosis in a patient.

43. A method of providing a therapeutic composition comprising:
administering an anti-hypertensive drug;
administering inhaled nitric oxide; and
reducing symptoms of oxidative stress and/or fibrosis in a patient.

44. The method of claim 41, wherein the anti-fibrotic drug is an IPF drug.

45. The method of claim 42, wherein the calcium channel blocker is nifedipine.

46. The method of claim 43, wherein the anti-hypertensive drug is riociguat.

47. A method of providing a therapeutic composition comprising:
administering a prostacyclin;
administering inhaled nitric oxide;
and allowing the nitric oxide to provide an additive effect to reduce symptoms of oxidative stress and/or fibrosis in a patient.

48. A method of providing a therapeutic composition comprising:
administering a caspase regulator;
administering inhaled nitric oxide; and
modulating cellular apoptosis in a patient.

49. A method for providing a therapeutic composition comprising:
identifying a mammal having or at risk of developing a haemolytic condition;
positioning a mammal for nitric oxide treatment;
administering exogenous nitric oxide; and
reducing symptoms of hemolysis in the mammal.

50. The method of claim 50, wherein the hemolysis resulted from venom.

51. The method of claim 50, wherein the venom is from a snake, scorpion, or sea anemone.

52. The method of claim 50, wherein the hemolysis resulted from a bacterial infection.

53. The method of claim 50, wherein the hemolysis is caused by proteolysis.

54. A method of providing a therapeutic composition comprising:
identifying a mammal having or at risk of developing an ischemic condition;
administering exogenous nitric oxide; and
administering a drug with the nitric oxide to modulate remote ischemic conditioning pathway.

55. The method of claim wherein exogenous NO is administered over approximately a 30 minute period at low dose effective to cause accumulation of hypoxia inducible factor(s) and PHDs to promote ROS signalling.

56. The method of claim 54, further comprising improving organ preservation by down regulating mitochondrial metabolic activity.

57. The method of claim 55, wherein modulating hypoxia inducible factor(s) causes erythropoietin production to stimulate red cell production.

58. The method of claim 1, further comprising modulating a platelet derived growth factor pathway to reduce symptoms of fibrosis in a patient.
